(19) United States
(12) Patent Application Publication
(10) Pub. No.: US 2002/0192273 A1
(43) Pub. Date: Dec. 19, 2002

(54) Therapeutic patch useful for the treatment of hemorrhoids

(76) Inventors: Teri Buseman, Minnetonka, MN (US); David Rolf, Eden Prairie, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/120,205

(22) Filed: Apr. 10, 2002

Related U.S. Application Data
(60) Provisional application No. 60/298,718, filed on Jun. 15, 2001.

(51) Int. Cl. A61K 31/137; A61K 9/70

(52) U.S. Cl. 424/449; 514/649

(57) ABSTRACT

The present invention provides an adhesive patch that includes a flexible backing having a front side and a back side. A therapeutic formulation is positioned on at least a portion of the front side of the backing, in at least a portion of the front side of the backing, or on and in at least a portion of the front side of the backing. The therapeutic formulation includes a vasoconstrictor, a solvent that dissolves the vasoconstrictor, and a pressure sensitive adhesive. The present invention also provides methods of medical use that employ the patch of the present invention. Such uses include, e.g., treating or preventing hemorrhoids in a mammal, providing relief from the discomfort associated with hemorrhoids, providing post-operative relief from discomfort associated with the surgical treatment of hemorrhoids, treating or preventing a bacterial infection associated with hemorrhoids, preventing a bacterial infection associated with the surgical treatment of hemorrhoids, absorbing exudate, blood, or a combination thereof from the region of the anus of a mammal inflicted with hemorrhoids, and absorbing exudate, blood, or a combination thereof from the region of the anus of a mammal during the post-operative treatment of hemorrhoids.

### BACKGROUND OF THE INVENTION

Hemorrhoids are a varicosity in the lower rectum or anus caused by congestion in the veins of the hemorrhoidal plexus. Hemorrhoids are an enlarged, swollen or dilated (varicose) vein situated at or near the anus. Mosby's Medical, Nursing, & Allied Health Dictionary, Kenneth Anderson, 5th Ed., St. Louis, Mo. (1998); and Stedman's Medical Dictionary, Illustrated, (25th Ed.), Williams & Wilkins: Baltimore (1990).

Hemorrhoids can be internal or external. Internal hemorrhoids occur higher up in the anal canal, beginning above the internal opening of the anus. http://www.hemcare.com/what.htm; and Mosby's Medical Encyclopedia, CD-Rom version 2.0 (1997). If they become large enough to protrude from the anus, they become squeezed and painful. Mosby's Medical Encyclopedia, CD-Rom version 2.0 (1997). Small internal hemorrhoids may bleed with bowel movements. Mosby's Medical Encyclopedia, CD-Rom version 2.0 (1997). Bleeding is the most common symptom of internal hemorrhoids, and often the only symptom in mild cases. http://www.hemcare.com/what.htm. Sometimes, internal hemorrhoids will come through the anal opening if straining occurs by the patient when making bowel movements. http://www.hemcare.com/what.htm. This is called a prolapsed internal hemorrhoid and when this occurs, it is often difficult to ease the hemorrhoidal tissue back into the rectum and is usually painful to the patient. http://www.hemcare.com/what.htm.

External hemorrhoids are visible hemorrhoids occurring outside the anal opening. http://www.hemcare.com/what.htm; Mosby's Medical Encyclopedia, CD-Rom version 2.0 (1997). External hemorrhoids are basically skin-covered veins that have ballooned and which appear blue. http://www.hemcare.com/what.htm. Usually, external hemorrhoids appear without any symptoms. http://www.hemcare.com/what.htm. When inflamed, however, external hemorrhoids become red and tender. http://www.hemcare.com/what.htm. External hemorrhoids are usually not painful, and bleeding does not occur unless a hemorrhoidal vein breaks or becomes blocked. Mosby's Medical Encyclopedia, CD-Rom version 2.0 (1997). If a blood clot forms inside an external hemorrhoid, the thrombosed external hemorrhoid can be felt as a firm, tender mass in the anal area, about the size of a pea that often causes the patient severe pain. http://www.hemcare.com/what.htm.

Hemorrhoids occur in about 89% of all Americans at some time in their lives and over two thirds of all healthy people reporting for physical examinations have hemorrhoids. http://www.hemcare.com/what.htm. Hemorrhoids are typically caused by straining to defecate, constipation, and/or too much sitting. Mosby's Medical Encyclopedia, CD-Rom version 2.0 (1997). Because pregnancy can help bring about hemorrhoids, pregnant women is advised to avoid constipation. Mosby's Medical Encyclopedia, CD-Rom version 2.0 (1997). When hemorrhoids become inflamed, the patient can suffer pain, bleeding, clots, and itching. University of Maryland Surgery Website (http://umm.drkoop.com/conditions/ency/article/002939.htm); and http://www.hemcare.com/what.htm. Unfortunately a hemorrhoidal condition only tends to worsen over the years. http://www.hemcare.com/what.htm.

Safe, gentle, and effective treatment of hemorrhoids are recommended as soon as they occur. Current treatments include the use of surface medication to lubricate and shrink the hemorrhoid reducing pain. Mosby's Medical Encyclopedia, CD-Rom version 2.0 (1997). http://www.hemcare.com/what.htm. Sitz baths and cold or hot packs are also soothing to the patient. Mosby's Medical Encyclopedia, CD-Rom version 2.0 (1997); and http://www.hemcare.com/what.htm.

Hemorrhoid removal may be recommended when non-surgical treatment (fiber rich diet, laxatives, stool softener, suppositories, medications, warm baths) does not provided adequate relief to the patient from persistent itching, anal bleeding, pain and blood clots (thrombosis of the hemorrhoids). University of Maryland Surgery Website (http://umm.drkoop.com/conditions/ency/article/002939.htm). Hemorrhoids maybe removed by either hardening the hemorrhoid by injection and then tying it off, or by surgical procedure. Mosby's Medical Encyclopedia, CD-Rom version 2.0 (1997). http://www.hemcare.com/what.htm. Tying off is increasingly the preferred treatment because it is simple, effective, and does not require anesthesia. Mosby's Medical Encyclopedia, CD-Rom version 2.0 (1997). http://www.hemcare.com/what.htm. In the tying off method, the hemorrhoid is grasped with a forceps and a rubber band is slipped over the enlarged part, causing the tissue to die and the hemorrhoid to fall off, usually within 1 week. Mosby's Medical Encyclopedia, CD-Rom version 2.0 (1997). http://www.hemcare.com/what.htm. Hemorrhoids may also be removed surgically while the patient is sleepy (sedated) and pain-free (local anesthesia or spinal anesthesia) or deep asleep and pain-free (general anesthesia). The enlarged vein (hemorrhoid) is removed and a gauze packing is inserted to reduce bleeding. University of Maryland Surgery Website (http://umm.drkoop.com/conditions/ency/article/002939.htm).

There are several drawbacks with the surgical procedures to remove hemorrhoids. The patient may experience considerable pain after surgery as the anus tightens and relaxes, wherein prescription medications would be needed to alleviate the pain. University of Maryland Surgery Website (http://umm.drkoop.com/conditions/ency/article/002939.htm) To avoid straining, stool softeners are typically used. University of Maryland Surgery Website (http://umm.drkoop.com/conditions/ency/article/002939.htm) The patient would need to avoid straining during bowel movements or urination. University of Maryland Surgery Website (http://umm.drkoop.com/conditions/ency/article/002939.htm) Complete recovery usually takes up to about 2 weeks. University of Maryland Surgery Website (http://umm.drkoop.com/conditions/ency/article/002939.htm). Additionally, the outcome is successful in only about 90% of the cases. University of Maryland Surgery Website (http://umm.drkoop.com/conditions/ency/article/002939.htm.

There are also risks associated with the surgical removal of hemorrhoids. Reactions to the anesthesia itself may cause difficulty in breathing. University of Maryland Surgery Website (http://umm.drkoop.com/conditions/ency/article/002939.htm) The risks associated with the surgery also include bleeding, infection and even the narrowing (stricture) of the anus. University of Maryland Surgery Website (http://umm.drkoop.com/conditions/ency/article/002939.htm. The costs of surgery varies significantly between surgeons, medical facilities, and regions of the country. Patients that are younger, more ill, or need more extensive surgery will require more intensive and expensive treatment. University of Maryland Surgery Website (http://umm.drkoop.com/conditions/ency/article/002939.htm). Surgery charges typically include surgeon's fees, anesthesiologist's fees, hospital care, operating room charges, medications, and additional charges. University of Maryland Surgery Website (http://umm.drkoop.com/conditions/ency/article/002939.htm. Additional charges may include surgeon assistant fees, treatment of complications, diagnostic procedures (such as blood or X-ray exams), medical supplies, and/or equipment use. University of Maryland Surgery Website (http://umm.drkoop.com/conditions/ency/article/002939.htm. As such, many individuals may prefer not to undergo the costly and risky medical procedure to remove the hemorrhoid.

There is a need therefore for methods and devices for treating patients inflicted with hemorrhoids that have minimum adverse effects; have maximum efficacy; are relatively inexpensive, safe, simple, and comfortable to use; administers to the skin an effective and known amount of vasoconstrictor; complies with FDA regulations; and/or absorbs any exudate, blood, or combination thereof from the hemorrhoidal tissue. The device can preferably have an overall yield of product that is higher than current devices, can preferably have an improved “holdout” of therapeutic formulation on the backing compared to current devices, and/or can have a lower degree of penetration of the therapeutic formulation in the backing of the device compared to known devices.

### SUMMARY OF THE INVENTION

The present invention provides a protective, adhesive skin patch useful for people inflicted with hemorrhoids. The adhesive patch of the present invention can be used to prevent or treat hemorrhoids, can be used to provide relief from the discomfort associated with hemorrhoids, can be used to prevent or treat a bacterial infection associated with hemorrhoids, and/or can be used to absorb exudate, blood, or a combination from the region of the anus. The adhesive skin patch administers to the region of the anus an effective and known amount of a vasoconstrictor. The adhesive skin patch maintains the adhesiveness of the adhesive and the stability of the vasoconstrictor over a prolonged period of time typically experienced in the manufacturing, packaging, shipping, and/or the storage of the adhesive patch.

The vasoconstrictor, solvent, and pressure sensitive adhesive are positioned on at least a portion of the adhesive patch, in at least a portion of the adhesive skin patch, or on and in at least a portion of the adhesive patch. Preferably, the vasoconstrictor, solvent, and pressure sensitive adhesive are partially embedded in at least a portion of the adhesive skin patch. Additionally, the adhesive skin patch complies with FDA regulations (e.g., 21 C.F.R. Chapter 1, Part 346—Anorectal Drug Products for Over-The-Counter Human Use).

The adhesive skin patch of the present invention can include a gel that is not water-based. The adhesive skin patch can include a backing that is treated with a sizing agent (e.g., a fluorocarbon solution, a silicone-containing compound, or a combination thereof). The sizing agent can be a hydrophobic sizing agent. The use of such backing can prevent immediate wick through and can maintain the hydrogel from penetrating the backing too quickly. In addition, the use of such backing can provide an adhesive skin patch with a higher yield improvement and superior holdout properties. The use of such backing can also obviate the need for a backing liner or a release liner. In such an embodiment, the adhesive skin patch can exist as a self wound adhesive patch.

The present invention provides an adhesive patch that includes a flexible backing having a front side and a back side. A therapeutic formulation is positioned on at least a portion of the front side of the backing, in at least a portion of the front side of the backing, or on and in at least a portion of the front side of the backing. The therapeutic formulation includes a vasoconstrictor, a solvent that dissolves the vasoconstrictor, and a pressure sensitive adhesive.

The present invention also provides a method for treating or preventing hemorrhoids in a mammal in need thereof. The method includes applying to the region of the anus of the mammal having the hemorrhoid or to the region of the anus of the mammal at risk thereof an adhesive patch of the present invention for a period of time effective to treat or prevent the hemorrhoid.

The present invention also provides a method for providing relief from the discomfort associated with hemorrhoids. The method includes applying to the region of the anus of a mammal having the hemorrhoid an adhesive patch of the present invention for a period of time effective to provide the relief.

The present invention also provides a method for providing post-operative relief from discomfort associated with the surgical treatment of hemorrhoids. The method includes applying to the region of the anus of the mammal an adhesive patch of the present invention for a period of time effective to provide the relief.

The present invention also provides a method for treating or preventing a bacterial infection associated with hemorrhoids. The method includes applying to the region of the anus of a mammal having the hemorrhoid or to the region of the anus of a mammal at risk thereof an adhesive patch of the present invention for a period of time effective to treat or prevent the bacterial infection.

The present invention also provides a method for treating or preventing a bacterial infection associated with the surgical treatment of hemorrhoids. The method includes applying to the region of the anus of the mammal an adhesive patch of the present invention for a period of time effective to treat or prevent the bacterial infection.

The present invention also provides a method for absorbing exudate, blood, or a combination thereof from the region of the anus of a mammal inflicted with hemorrhoids. The method includes applying to the region of the anus of the mammal an adhesive patch of the present invention for a period of time effective to absorb the exudate, blood, or the combination thereof.

The present invention also provides a method for absorbing exudate, blood, or a combination thereof from the region of the anus of a mammal during the post-operative treatment of hemorrhoids. The method includes applying to the region of the anus of the mammal an adhesive patch of the present invention for a period of time effective to absorb the exudate, blood, or the combination thereof.

### BRIEF DESCRIPTION OF THE FIGURES

FIG. 1 illustrates the front side of an adhesive patch of the present invention.

FIG. 2 illustrates the back side of an adhesive patch of the present invention.

FIG. 3 illustrates the front side of an adhesive patch of the present invention with a release liner attached to the patch.

FIG. 4 illustrates the back side of an adhesive patch with a release liner attached to the patch.

FIG. 5 illustrates the back side of an adhesive patch of the present invention with a release liner attached to the patch, wherein the patch is partially detached from the release liner.

FIG. 6 illustrates the back side of an adhesive patch of the present invention with a release liner attached to the patch, wherein the patch is partially detached from the release liner.

FIG. 7 illustrates a top view of a specific patch of the present invention.

FIG. 8 illustrates a top view of a specific patch of the present invention.

FIG. 9 illustrates an enlarged cross-sectional view of specific patch of the present invention.

FIG. 10 illustrates a specific adhesive skin patch of the present invention.

### DETAILED DESCRIPTION OF THE INVENTION

The present invention provides a unique adhesive vehicle. The vehicle has pressure sensitive adhesive qualities due to its composition and viscoelastic nature. The adhesive is hydrophilic and therefore water can dissolve into or evaporate from the adhesive, depending on the conditions to which it is exposed. This water exchange capability implies that if the adhesive is on a suitably porous backing and is applied to the skin, it will not be occlusive as most drug delivery patches are. The occlusive nature of conventional drug delivery patches is thought to play an important role in enhancing drug absorption, but also often results in greater incidence of skin irritation. The relatively low occlusiveness of the present invention can be envisioned to be a special adhesive ointment or gel which is water-breathable, such as a water washable or water soluble ointment or gel.

The present invention provides an ointment or gel on a backing. The ointment or gel includes an effective, known, and safe amount of a vasoconstrictor that is useful in the treatment of hemorrhoids. The backing is pliable and/or stretchable. Since the backing can be porous and/or vapor permeable, many consumers typically refer to the device as a “patch,” a “skin patch,” or an “adhesive skin patch.” As such, the device (i.e., the ointment or gel on the backing) will herein be referred to as a patch, a skin patch, an adhesive skin patch and/or as a hemorrhoidal patch. It is appreciated that those skilled in the art understand that the term “patch” is used to refer to the device and is not otherwise limiting in any manner.

The present invention provides a water soluble or a water insoluble, protective, adhesive patch useful for treating or preventing hemorrhoids. The patch administers to the skin an effective and known amount of a vasoconstrictor. The patch maintains the adhesiveness of the adhesive and the stability of the vasoconstrictor over a prolonged period of time typically experienced in the manufacturing, packaging, shipping, and/or the storage of the patch. The patch complies with FDA regulations (e.g., 21 C.F.R. Chapter 1, Part 346—Anorectal Drug Products for Over-The-Counter Human Use). The adhesive patch of the present invention can include a gel that is not water-based. The adhesive patch includes a backing that is treated with a sizing agent (e.g., a fluorocarbon solution, a silicone-containing compound, or a combination thereof). The use of such backing prevents immediate wick through and maintains the hydrogel from penetrating the backing too quickly. In addition, the use of such backing provides a patch with a higher yield improvement and superior holdout properties. The use of such backing also obviates the need for a backing liner or a release liner. In such an embodiment, the adhesive patch can exist as a self wound adhesive patch.

As used herein, “holdout” refers to the physical properties of a backing, relating to the ability of a specific class of gels or ointments to penetrate, cross-link, wet, and/or cure within the matrix of the backing. A specific class of gels or ointments may or may not be able to penetrate a given backing. Upon penetration of a gel or ointment into a backing, the gel or ointment will cross-link, wet, or cure in the backing. As such, the holdout properties are a degree of the ability of the gel or ointment to affect the degree of penetration, cross-linking, wetting, and/or curing within the matrix of the backing. Those backings with superior holdout properties will typically prevent, decrease, or lessen the likelihood of the ointment or gel from wetting the backing; will typically increase the likelihood of the ointment or gel to cross-link within the matrix of the backing; will typically increase the likelihood of the ointment or gel to cure within the matrix of the backing; and/or will typically prevent, decrease, or increase the likelihood of the ointment or gel to partially penetrate the matrix of the backing.

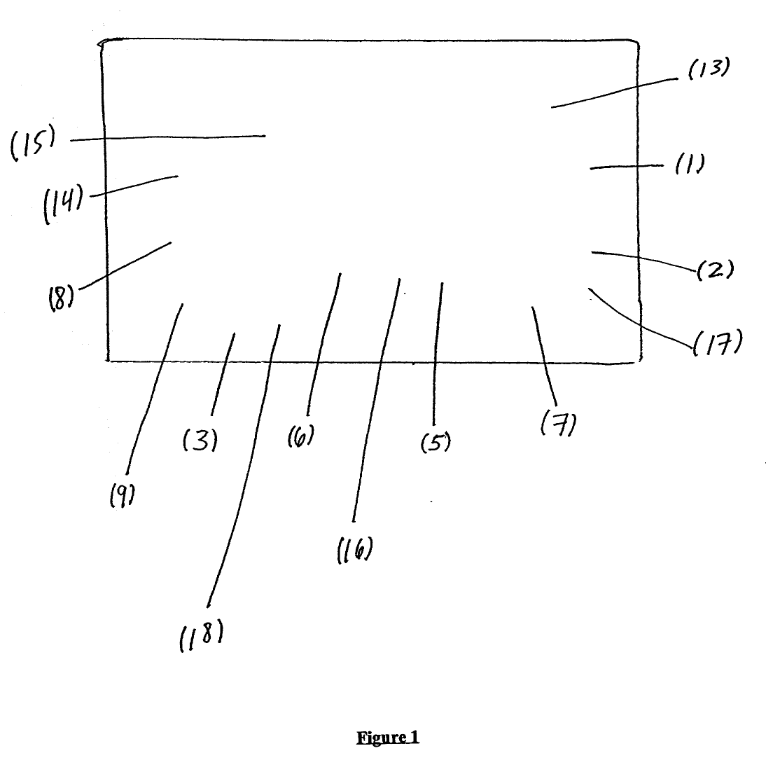

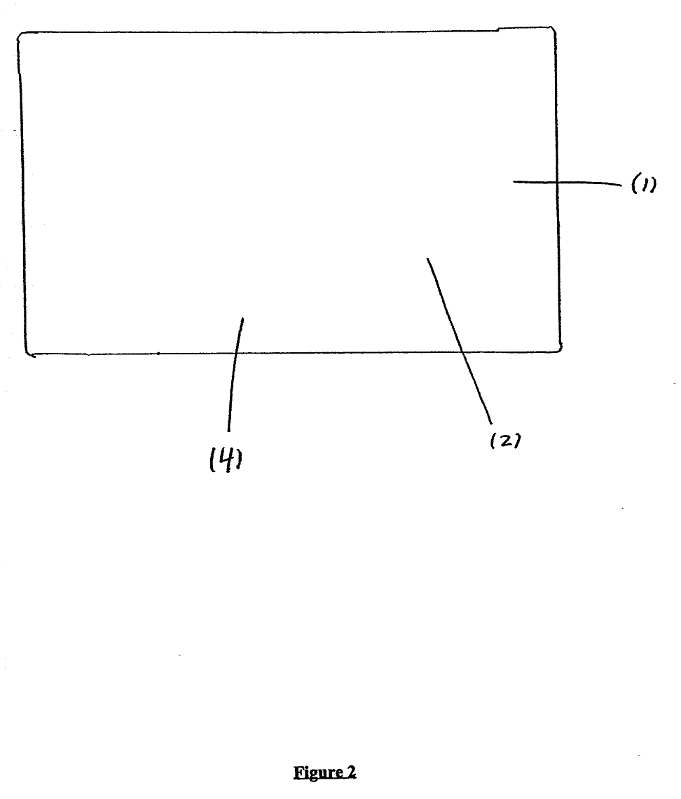

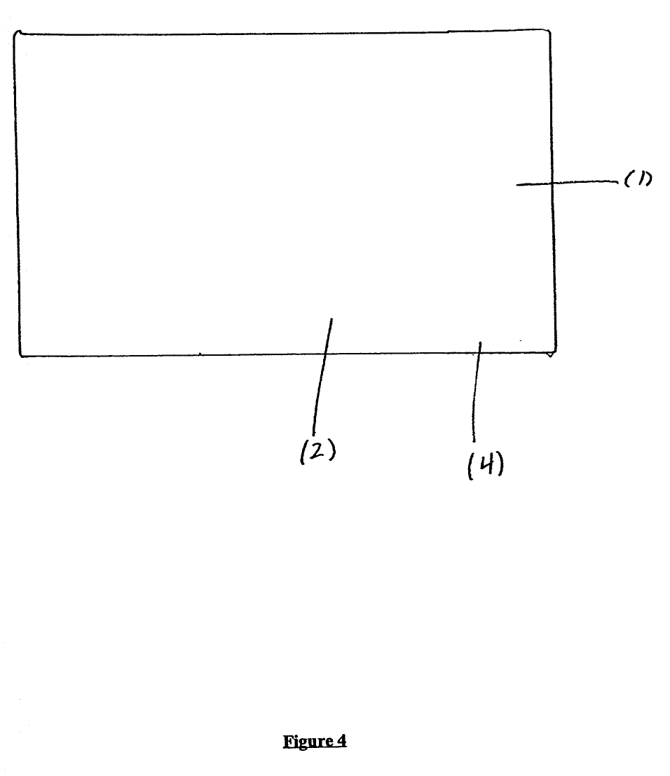

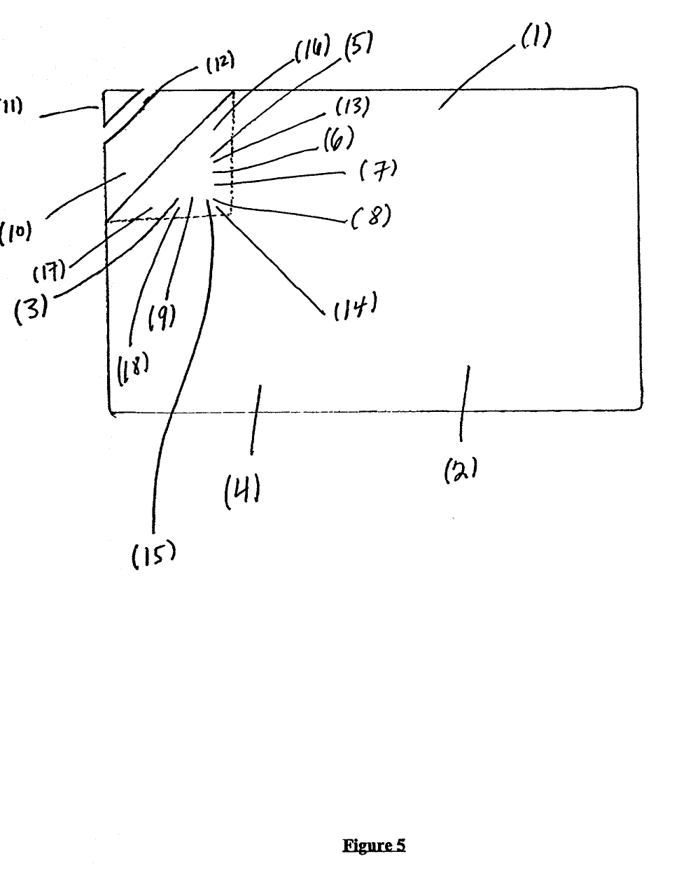

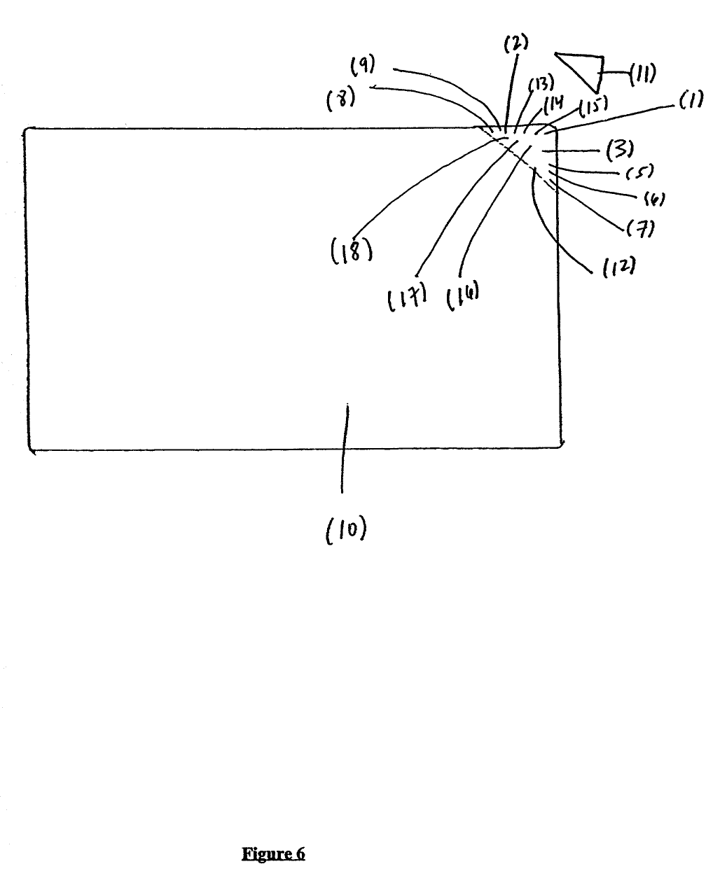

Referring to FIGS. 1-10, an adhesive patch 1 of the present invention is provided. The adhesive patch 1 includes a therapeutic formulation 5 located on at least a portion of the front side 3 of the backing 2, in at least a portion of the front side 3 of the backing 2, or on and in at least a portion of the front side 3 of the backing 2. Preferably, the therapeutic formulation 5 is partially embedded in at least a portion of the front side 3 of the backing 2. In addition to being located in at least a portion of the front side 3 of the backing 2, the therapeutic formulation 5 is located on a portion of the surface of front side 3 of the backing 2. Preferably, the therapeutic formulation 5 is located on the entire surface of the front side 3 of the backing 2.

Backing

The backing 2 is defined by a front side 3 (the side exposed to the skin during use) and a back side 4 (the side exposed to the environment during use). The backing 2 should be nonirritating to human skin. The backing 2 is a self-supporting sheet of water soluble or water insoluble, polymeric or natural material that provides strength and integrity for the therapeutic formulation 5. The backing 2 of the adhesive patch 1 can be vapor permeable. The backing 2 can also be porous, since porosity provides openings for receiving the therapeutic formulation 5 and it helps to assure that the adhesive skin patch 1 is vapor permeable. Specifically, the backing 2 can retain the therapeutic formulation 5 while allowing moisture from the skin to pass. The backing 2 can have any suitable thickness, provided the suitable thickness allows for a flexible, bendable, pliable, vapor permeable, and/or a stretchable sheet of water insoluble porous material. Specifically, the thickness of the backing 2 can be about 0.001 mm to about 5.0 mm, about 0.001 mm to about 3.0 mm, or about 0.025 mm to about 1.25 mm.

The backing 2 can be manufactured from any suitable material, provided the suitable material can form a flexible, bendable, pliable, and/or stretchable backing 2. The backing 2 includes a flexible porous sheet of water soluble or water insoluble material that provides support for the adhesive skin patch 1. The backing 2 can include water soluble or water insoluble polymeric fibers, a porous film, or any other kind of matrix with spaces within the matrix. A specific backing 2 is a lightweight, porous, pliable strip composed of a nonwoven fabric of polymeric or natural fibers such as polyester, cotton or cellulose fibers bonded together with a sizing resin. The backing 2 can be woven or nonwoven. Preferably, the backing 2 includes nonwoven fabric. Specifically, the backing 2 can include polyester, polyurethane, polyolefin, polyamide fibers, natural fibers, cotton fibers, polycellulose fibers, or any mixture thereof. Additional stable, water insoluble flexible sheet materials and methods for manufacturing the suitable backings 2 are disclosed, e.g., in U.S. Pat. No. 4,675,009; U.S. Pat. No. 5,536,263; U.S. Pat. No. 4,696,854; U.S. Pat. No. 5,741,510, and references cited therein, and are suitable as backings 2 according to the present invention. The infusion of the therapeutic formulation 5 into the backing 2 can be accomplished, e.g., with the use of a continuous process mixer, as disclosed, e.g., in U.S. Pat. No. 5,536,263, and references cited therein; or as discussed herein.

Alternatively, the backing 2 can be a non-woven backing 2 that is treated by coating: the front side 3 of the backing 2, the back side 4 of the backing 2, or both the front side 3 and back side 4 of the backing 2; with a silicone-containing compound. Suitable silicone-containing compounds include, e.g., polydimethyl siloxanes, dialkylsiloxanes, dimethylsiloxo vinyl alkenes, dialkylsiloxo vinyl alkenes, dimethylsiloxo acrylates, dialkylsiloxo acrylates, vinyl terminated polydimethylsiloxane, and vinyl terminated polydialkylsiloxane. The exemplary silicone-containing compounds are commercially available from, e.g., Goldschmidt Chemical Corp. (Essen, Germany); GE Silicones (Waterford, N.Y.); Wacker Silicone Corp. (Adrian, Mich.); and Dow Corning Corp. (Midland, Mich.).

The backing 2 can be manufactured from a suitable non-woven fabric that is commercially available from, e.g., Freudenberg Faservliesstoffe KG (Weinham, Germany); Sontara Technologies (division of DuPont Corporation) (Old Hickory, Tenn.); Lystil S. A. (Brignoud Cedex, France); Dexter Nonwovens (Windsor Locks, Conn.); and Chicopee (New Brusnwick, N.J.). Other commercial vendors that supply suitable non-woven fabrics can be found at the Technical Textile website (http://www.technical-textiles.net/technical-textiles-index/orgL.htm).

It has surprisingly been discovered that the use of a treated backing, such as a fluorocarbon treated non-woven backing, typically increases the yield of an adhesive patch. The use of a backing material that has been treated with a sizing agent allows for the effective control of the rate of penetration, such that the gel or ointment has solidified after it has begun to penetrate the backing, but before it has passed completely through the backing. In addition, the use of a backing material that has been treated with a sizing agent allows for the effective control of the depth to which the ointment or gel will easily penetrate before solidifying. It has surprisingly been discovered that increasing the control of the rate at which the ointment or gel penetrates the backing typically improves the overall yield of the production process by reducing the amount of material which must be discarded because the back side of the backing has become too tacky for either processing or for consumer acceptance.

At least a portion of the backing 2 can be treated with a sizing agent 8 such that the portion of the backing 2 that is treated with the sizing agent 8 has a surface energy of about 20 dynes/cm² to about 65 dynes/cm². Specifically, the portion of the backing 2 that is treated with the sizing agent 8 can have a surface energy of about 27 dynes/cm² to about 56 dynes/cm². The sizing agent 8 lowers the surface energy of the portion of the backing 2 that is treated with the sizing agent 8. Any suitable sizing agent 8 can be employed, provided the portion of the backing 2 that is treated with the sizing agent 8 has a surface energy of about 20 dynes/cm² to about 65 dynes/cm². Suitable sizing agents 8 include, e.g., fluorocarbon solutions, silicone-containing compounds, and combinations thereof. Specifically, the backing 2 can be a non-woven backing 2 that is treated with a fluorocarbon. For example, the fluorocarbon treated backing 2 can be, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, or Vilmed M1578 FH; which are all commercially available from Freudenberg Faservliesstoffe KG (Weinham, Germany). Alternatively, the silicone treated backing 2 can be a non-woven backing 2 that is coated with one or more silicone-containing compounds, e.g., a polydimethyl siloxane, a dialkylsiloxane, a dimethylsiloxo vinyl alkene, a dialkylsiloxo vinyl alkenes, a dimethylsiloxo acrylate, a dialkylsiloxo acrylate, a vinyl terminated polydimethylsiloxane, and a vinyl terminated polydialkylsiloxane.

At least a portion of the backing 2 can be treated with the sizing agent 8. The portion of the backing 2 that is treated with the sizing agent 8 can be that portion of the backing 2 that can typically include the therapeutic formulation 5. The entire surface of the front side 3 of the backing 2 can be treated with the sizing agent 8 or a portion of the surface of the front side 3 of the backing 2 can be treated with the sizing agent 8. Preferably, the entire surface of the front side 3 of the backing 2 can be treated with the sizing agent 8. In addition to the surface of the front side 3 of the backing 2 being treated with the sizing agent 8, the sizing agent 8 can penetrate at least a portion of the underlying surface (e.g., one-tenth to about nine-tenths the thickness, or about one-fourth to about nine-tenths the thickness) of the backing 2. Specifically, the sizing agent 8 can penetrate the entire underlying surface of the backing 2.

Suitable fluorocarbon treated backings 2 include, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, and Vilmed M1578 FH; which are all commercially available from Freudenberg Faservliesstoffe KG (Weinham, Germany).

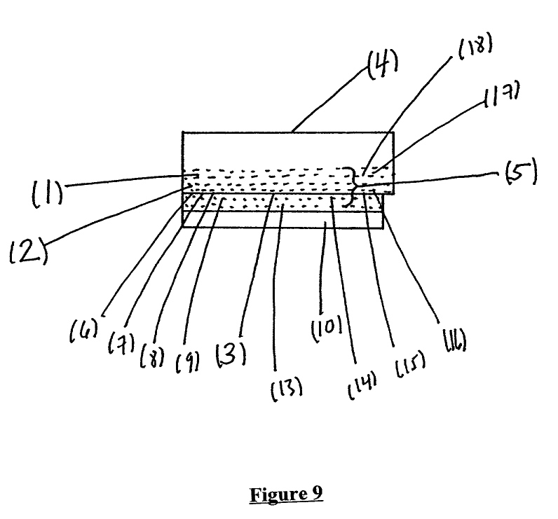

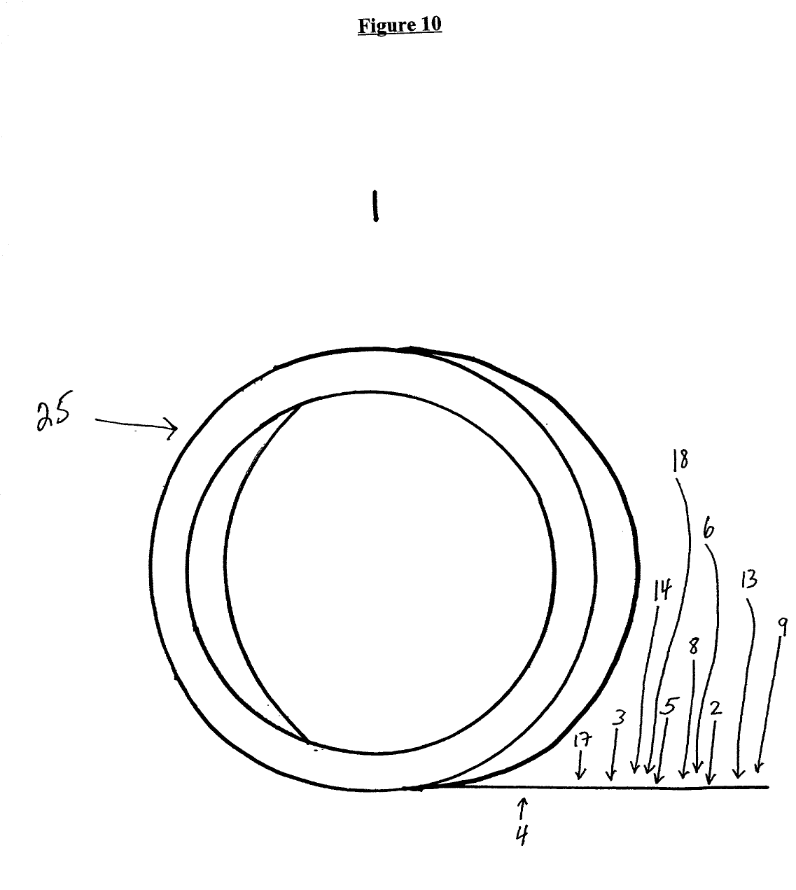

As shown in FIGS. 1-6 and 9-10, the backing 2 includes a front side 3 and a back side 4. The adhesive skin patch 1 includes a therapeutic formulation 5 located in at least a portion of the front side 3 of the backing 2, on at least a portion of the front side 3 of the backing 2, or on and in at least a portion of the front side 3 of the backing 2. As such, the therapeutic formulation 5 can be located on the entire surface of the front side 3 of the backing 2 or the therapeutic formulation 5 can be located on a portion of the surface of the front side 3 of the backing 2. Preferably, the therapeutic formulation 5 can be located on the entire surface of the front side 3 of the backing 2. In addition to being located on the surface of the front side 3 of the backing 2, the therapeutic formulation 5 can be located in at least a portion of the underlying surface of the front side 3 of the backing 2 (i.e., the therapeutic formulation 5 can be partially embedded into the backing 2). As shown in FIG. 9, the therapeutic formulation 5 can penetrate a substantial portion of the front side 3 of the backing 2, as disclosed, e.g., in U.S. Pat. No. 5,536,263, and references cited therein. For example, the therapeutic formulation 5 can penetrate about one-tenth to about nine-tenths the thickness of the backing 2, or about one-fourth to about nine-tenths the thickness of the backing 2. As such, the therapeutic formulation 5 can be partially embedded into the backing 2. Preferably, the therapeutic formulation 5 can be located on the entire front side 3 of the backing 2 and partially in the front side 3 of the backing 2 (i.e., the therapeutic formulation 5 is partially embedded into the backing 2). Alternatively, a portion of the front side 3 of the backing 2 can include the therapeutic formulation 5 and other portions of the front side 3 of the backing 2 can include any combination of the pressure sensitive adhesive 14, vasoconstrictor 15, and solvent 13. For example, a central circular portion of the front side 3 of the backing 2 can include the therapeutic formulation 5 and solvent 13 while the remaining portions of the front side 3 of the backing 2 include only the pressure sensitive adhesive 14. The therapeutic formulation 5, when partially embedded into the front side 3 of the backing 2, imparts strength and structure into the adhesive patch 1. For example, when the therapeutic formulation 5 is partially embedded into the backing 2, the likelihood that the adhesive patch 1 will tear apart when separated from the release liner 10 or when removed from the skin after use, is minimized. When the adhesive skin patch 1 is placed upon the skin of a patient (e.g., human), the therapeutic formulation 5 can be in continuous contact with the skin surface of the patient.

Preferably, the adhesive skin patch 1, upon contact with skin, will allow the skin to breathe. More preferably, the adhesive skin patch 1, upon prolonged contact with skin, will hold in place the therapeutic formulation 5 and will permit the skin to breathe over prolonged periods of time typically experienced with the use of the patch, e.g., up to about 24 hours, up to about 12 hours, up to about 8 hours, or up to about 6 hours.

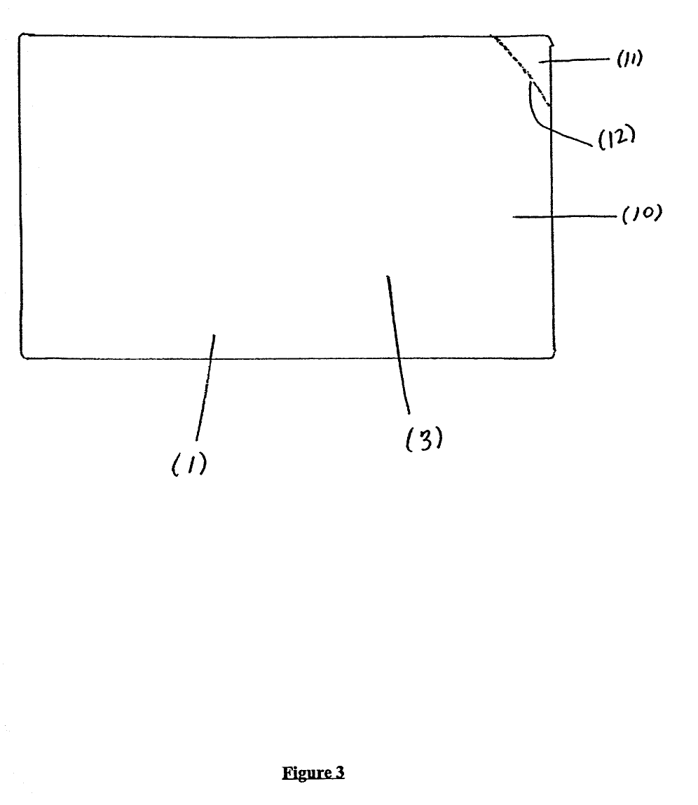

As shown in FIGS. 3-6 and 9, the adhesive skin patch 1 can be reversibly attached to a release liner 10. The release liner 10 helps to maintain the adhesiveness of the adhesive skin patch 1 prior to use, such as during manufacturing, packaging, shipping, and/or storage. Any suitable release liner 10 can be employed for use in the present invention. Suitable release liners 10 are readily known to those of skill in the art. See, e.g., U.S. Pat. No. 4,675,009; U.S. Pat. No. 5,536,263; U.S. Pat. No. 4,696,854; U.S. Pat. No. 5,741,510, and references cited therein for further descriptions of release liners 10 useful in the present invention. The release liner 10 can include a perforation 12 that allows the tab section 11 of the release liner 10 to be removed (see, FIGS. 3, 5, and 6). Removal of the tab section 11 of the release liner 10 allows the adhesive skin patch 1 to be removed from the release liner 10 with relative ease.

Vasoconstrictor

As used herein, a “vasoconstrictor” refers to a substance or agent that promotes the constriction of blood vessels in hemorrhoidal tissue. Mosby's Medical Nursing, & Allied Health Dictionary, Kenneth Anderson, 5th Ed., St. Louis, Mo. (1998). The vasoconstrictor is a substance that causes temporary constriction of blood vessels. 21 C.F.R. Chapter 1, Part 346—Anorectal Drug Products for Over-The-Counter Human Use. Any suitable vasoconstrictor 15 can be employed, provided the vasoconstrictor 15 effectively promotes the constriction of blood vessels of hemorrhoidal tissue (i.e., the vasoconstrictor 15 shrinks the hemorrhoidal tissue) and the vasoconstrictor 15 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Suitable vasoconstrictors 15 are disclosed, e.g., in Physician's Desk Reference (PDR), Medical Economics Company (Montvale, N.J.), (53rd Ed.), 1999; Mayo Medical Center Formulary Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; 21 C.F.R. Chapter 1, Part 346—Anorectal Drug Products for Over-The-Counter Human Use; and references cited therein.

Any suitable vasoconstrictor 15 can be employed provided the vasoconstrictor 15 effectively promotes the constriction of blood vessels of hemorrhoidal tissue (i.e., the vasoconstrictor 15 shrinks the hemorrhoidal tissue) and the vasoconstrictor 15 remains stable in the therapeutic formulation 5. Specific vasoconstrictors 15 include, e.g., ephedrine, epinephrine, phenylephrine, a pharmaceutically acceptable salt thereof, or a combination thereof. More specifically, the vasoconstrictor 15 can be ephedrine sulfate, epinephrine, epinephrine hydrochloride, phenylephrine hydrochloride, or a combination thereof.

The vasoconstrictor 15 can be present in any appropriate and suitable amount. Specifically, the vasoconstrictor 15 can be present in about 0.01 wt. % to about 99.9 wt. % of the therapeutic formulation 5. Typically, the amount of vasoconstrictor 15 present in the therapeutic formulation 5 will depend upon the specific compound or compounds employed as the vasoconstrictor 15. Preferably, the amount of vasoconstrictor 15 will comply with FDA regulations (e.g., C.F.R. Chapter 1, Part 346—Anorectal Drug Products for Over-The-Counter Human Use).

The amount of vasoconstrictor 15 present in the therapeutic formulation 5 will typically depend upon the specific compound or compounds employed as the vasoconstrictor 15. The vasoconstrictor 15 can typically be present up to about 99.9 wt. % of the therapeutic formulation 5, up to about 10.0 wt. % of the therapeutic formulation 5, up to 2.0 wt. % of the therapeutic formulation 5, up to about 1.0 wt. % of the therapeutic formulation 5, up to about 0.1 wt. % of the therapeutic formulation 5, or up to about 0.01 wt. % of the therapeutic formulation 5.

In one embodiment of the present invention, ephedrine sulfate can be present up to about 1.25 wt. % of the therapeutic formulation 5; epinephrine can be present up to about 0.01 wt. % of the therapeutic formulation 5; epinephrine hydrochloride can be present up to about 0.01 wt. % of the therapeutic formulation 5; and/or phenylephrine hydrochloride can be present up to about 0.30 wt. % of the therapeutic formulation 5.

As disclosed in as disclosed in C.F.R. Chapter 1, Part 346—Anorectal Drug Products for Over-The-Counter Human Use, ephedrine sulfate can be present in about 0.1 wt. % of the therapeutic formulation 5 to about 1.25 wt. % of the therapeutic formulation 5; epinephrine can be present in about 0.005 wt. % of the therapeutic formulation 5 to about 0.01 wt. % of the therapeutic formulation 5; epinephrine hydrochloride can be present in about 0.005 wt. % of the therapeutic formulation 5 to about 0.01 wt. % of the therapeutic formulation 5; and/or phenylephrine hydrochloride can be present in about 0.20 wt. % of the therapeutic formulation 5 to about 0.30 wt. % of the therapeutic formulation 5.

The adhesive skin patch 1 includes a vasoconstrictor 15 located in at least a portion of the front side 3 of the backing 2, on at least a portion of the front side 3 of the backing 2, or on and in at least a portion of the front side 3 of the backing 2. As such, the vasoconstrictor 15 can be located on the entire surface of the front side 3 of the backing 2 or the vasoconstrictor 15 can be located on a portion of the surface of the front side 3 of the backing 2. Preferably, the vasoconstrictor 15 can be located on the entire surface of the front side 3 of the backing 2.

In addition to being located on the surface of the front side 3 of the backing 2, the vasoconstrictor 15 can be located in at least a portion of the underlying surface of the front side 3 of the backing 2 (i.e., the vasoconstrictor 15 can be partially embedded into the backing 2). As shown in FIG. 9, the vasoconstrictor 15 can penetrate a substantial portion of the front side 3 of the backing 2, as disclosed, e.g., in U.S. Pat. No. 5,536,263, and references cited therein. For example, the vasoconstrictor 15 can penetrate about one-tenth to about nine-tenths the thickness of the backing 2, or about one-fourth to about nine-tenths the thickness of the backing 2. As such, the vasoconstrictor 15 can be partially embedded into the backing 2.

Preferably, the vasoconstrictor 15 can be located on the entire front side 3 of the backing 2 and partially in the front side 3 of the backing 2 (i.e., the vasoconstrictor 15 is partially embedded into the backing 2). Alternatively, a portion of the front side 3 of the backing 2 can include the vasoconstrictor 15 and other portions of the front side 3 of the backing 2 can include any combination of the pressure sensitive adhesive 14 and solvent 13. For example, a central circular portion of the front side 3 of the backing 2 can include the vasoconstrictor 15 while the remaining portions of the front side 3 of the backing 2 include only the pressure sensitive adhesive 14 and solvent 13. When the adhesive skin patch 1 is placed upon the skin of a patient (e.g., human), the vasoconstrictor 15 can be in continuous contact with the skin surface of the patient.

As used herein, “hemorrhoid” refers to a variscosity in the lower rectum or anus caused by congestion in the veins of the hemorrhoidal plexus. Hemorrhoids are a enlarged, swollen or dilated (variscose) vein situated at or near the anus.

As used herein, “treat” or “treating” refers to providing relief of one or more of the symptoms associated with hemorrhoids; or the diminishing or lessening of any one or more of the symptoms associated with hemorrhoids. The symptoms typically encountered with hemorrhoids include, e.g., burning, itching, swelling, irritation, soreness, and/or inflammation of hemorrhoidal tissue. As such, the treatment of hemorrhoids can include the constriction of the blood vessels of the hemorrhoidal tissue, thereby effectively shrinking the hemorrhoid. Additionally, the treatment of hemorrhoids can include providing relief from the discomfort or pain from the burning, itching, swelling, irritation, soreness, and/or inflammation of hemorrhoidal tissue.

Solvent

The solvent 13 can act as a carrier for, and preferably can dissolve, the vasoconstrictor 15 and/or the pressure sensitive adhesive 14. Any suitable solvent 13 can be employed, provided the solvent 13 effectively dissolves the vasoconstrictor 15 and/or the pressure sensitive adhesive 14 and the solvent 13 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

The solvent 13 can include one or more organic compounds, one or more inorganic compounds, or mixtures thereof. Preferably, the solvent 13 will include one or more organic compounds, e.g., esters, terpenes, alcohols, ketones, aldehydes, fatty acids, partially or fully esterified fatty acids, wherein the structures are cyclic, non cylcic (e.g., alkyl), alicyclic (i.e., a bridged ring compound), or aromatic, as well as organic compounds having combinations of these functional groups. Suitable exemplary solvents 13 are disclosed, e.g., in Aldrich Handbook of Fine Chemicals, 2000-2001 (Milwaukee, Wis.).

Preferably, the solvent 13 includes a polyhydric alcohol, water, or a combination thereof. The polyhydric alcohol can be propylene glycol, ethylene glycol, triethylene glycol, or a combination thereof Additional suitable solvents 13 include, e.g., glycerin; triacetin; diethylene glycol methyl ether; diethylene glycol methyl ether acetate; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; a pharmaceutically acceptable salt thereof; or a combination thereof.

The solvent 13 can be employed in any suitable amount, provided the amount of solvent 13 is effective to dissolve the vasoconstrictor 15 and/or the pressure sensitive pressure sensitive adhesive 14 and the effective amount of solvent 13 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the solvent 13 can be present in about 1.0 wt % to about 70.0 wt. %; in about 3.0 wt % to about 50.0 wt. %; or in about 5 wt. % to about 30 wt. % of the therapeutic formulation 5. Typically, the amount of solvent 13 will depend on the compound or compounds employed as the solvent 13. For example, a polyhydric alcohol can be present up to about 70 wt. % of the therapeutic formulation 5; or in about 20.0 wt. % to about 60.0 wt. % of the therapeutic formulation 5; and water can be present in about 2.0 wt. % to about 50.0 wt. % of the therapeutic formulation 5.

The solvent 13 can be located in at least a portion of the front side 3 of the backing 2, on at least a portion of the front side 3 of the backing 2, or on and in at least a portion of the front side 3 of the backing 2. As such, the solvent 13 can be located on the entire surface of the front side 3 of the backing 2 or the solvent 13 can be located on a portion of the surface of the front side 3 of the backing 2. Preferably, the solvent 13 can be located on the entire surface of the front side 3 of the backing 2. In addition to being located on the surface of the front side 3 of the backing 2, the solvent 13 can be located in at least a portion of the underlying surface of the front side 3 of the backing 2 (i.e., the solvent 13 can be partially embedded into the backing 2).

As shown in FIG. 9, the solvent 13 can penetrate a substantial portion of the front side 3 of the backing 2, as disclosed, e.g., in U.S. Pat. No. 5,536,263, and references cited therein. For example, the solvent 13 can penetrate about one-tenth to about nine-tenths the thickness of the backing 2, or about one-fourth to about nine-tenths the thickness of the backing 2. As such, the solvent 13 can be partially embedded into the backing 2. Preferably, the solvent 13 can be located on the entire front side 3 of the backing 2 and partially in the front side 3 of the backing 2 (i.e., the solvent 13 is partially embedded into the backing 2). Alternatively, a portion of the front side 3 of the backing 2 can include the solvent 13 and other portions of the front side 3 of the backing 2 can include any combination of the pressure sensitive adhesive 14 and vasoconstrictor 15. When the adhesive skin patch 1 is placed upon the skin of a patient (e.g., human), the solvent 13 can be in continuous contact with the skin surface of the patient.

Pressure Sensitive Adhesive

Any suitable pressure sensitive pressure sensitive adhesive 14 can be employed, provided the pressure sensitive pressure sensitive adhesive 14 provides the requisite adhesiveness to the adhesive skin patch 1 and the pressure sensitive adhesive 14 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. It is appreciated that the suitable pressure sensitive adhesives 14 are known to those skilled in the art. Suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably the pressure sensitive adhesive 14 is an acrylic ester copolymer.

Any suitable amount of pressure sensitive adhesive 14 can be employed, provided the amount of pressure sensitive adhesive 14 effectively provides the requisite adhesiveness to the adhesive skin patch 1 and the effective amount of the pressure sensitive adhesive 14 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The therapeutic formulation 5 can include a pressure sensitive adhesive 14 in about 0.1 wt. % to about 50 wt. %, in about 0.5 wt. % to about 10.0 wt. %, or in about 1.0 wt. % to about 15.0 wt. % of the therapeutic formulation 5. Typically, the suitable amount of pressure sensitive adhesive 14 will depend upon the specific pressure sensitive adhesive 14 employed. For example, the pressure sensitive adhesive 14 can include one or more acrylic ester copolymers. Each of the one or more acrylic ester copolymers can be present up to about 20.0 wt. % of the therapeutic formulation 5. Specifically, each of the acrylic ester copolymers can be present up to about 40.0 wt. % of the therapeutic formulation 5, or up to about 30.0 wt. % of the therapeutic formulation 5. More specifically, all of the one or more acrylic ester copolymers, when combined, can be present in about 3.0 wt. % to about 40.0 wt. % of the therapeutic formulation 5, or in about 5.0 wt. % to about 30.0 wt. % of the therapeutic formulation 5. As such, the total amount of acrylic ester copolymers can be about 3.0 wt. % to about 40.0 wt. % of the therapeutic formulation 5, or about 5.0 wt. % to about 30.0 wt. % of the therapeutic formulation 5.

Alternatively, the pressure sensitive adhesive 14 can include a hot melt pressure sensitive adhesive 14 or solvent based pressure sensitive adhesive 14 (e.g., polyacrylate, polyisobutylene, and polybutene), rubber, silicone based pressure sensitive adhesives 14 (e.g., polydimethylsiloxane and resin mixtures), polystyrene-polybutadiene-polystyrene, polystyrene-polyisoprene-polystyrene, polystyrene-poly(ethylene-butylene)-polystyrene block polymers, or any combination thereof. In addition, the adhesive 14 can include a resin emulsion adhesive, wherein the resin emulsion adhesive can include vinyl acetate resin, acrylic ester copolymer, vinyl acetate/diocyl maleate copolymer, acrylic copolymer, or any combination thereof.

Other suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein.

The pressure sensitive adhesive 14 can be located in at least a portion of the front side 3 of the backing 2, on at least a portion of the front side 3 of the backing 2, or on and in at least a portion of the front side 3 of the backing 2. As such, the pressure sensitive adhesive 14 can be located on the entire surface of the front side 3 of the backing 2 or the pressure sensitive adhesive 14 can be located on a portion of the surface of the front side 3 of the backing 2. Preferably, the pressure sensitive adhesive 14 can be located on the entire surface of the front side 3 of the backing 2. In addition to being located on the surface of the front side 3 of the backing 2, the pressure sensitive adhesive 14 can be located in at least a portion of the underlying surface of the front side 3 of the backing 2 (i.e., the pressure sensitive adhesive 14 can be partially embedded into the backing 2). As shown in FIG. 9, the pressure sensitive adhesive 14 can penetrate a substantial portion of the front side 3 of the backing 2, as disclosed, e.g., in U.S. Pat. No. 5,536,263, and references cited therein. For example, the pressure sensitive adhesive 14 can penetrate about one-tenth to about nine-tenths the thickness of the backing 2, or about one-fourth to about nine-tenths the thickness of the backing 2. As such, the pressure sensitive adhesive 14 can be partially embedded into the backing 2. Preferably, the pressure sensitive adhesive 14 can be located on the entire front side 3 of the backing 2 and partially in the front side 3 of the backing 2 (i.e., the pressure sensitive adhesive 14 is partially embedded into the backing 2). Alternatively, a portion of the front side 3 of the backing 2 can include the pressure sensitive adhesive 14 and other portions of the front side 3 of the backing 2 can include any combination of the solvent 13 and vasoconstrictor 15. The pressure sensitive adhesive 14, being partially embedded into the front side 3 of the backing 2, imparts strength and structure into the adhesive patch 1. For example, when the pressure sensitive adhesive 14 is partially embedded into the backing 2, the likelihood that the adhesive patch 1 will tear apart when separated from the release liner 10 or when removed from the skin after use, is minimized. When the adhesive skin patch 1 is placed upon the skin of a patient (e.g., human), the pressure sensitive adhesives 14 can be in continuous contact with the skin surface of the patient.

Emulsifier

The therapeutic formulation 5 or pressure sensitive adhesive 14 can optionally include a compound that emulsifies the therapeutic formulation 5 or the pressure sensitive adhesive 14. One suitable compound that effectively emulsifies the therapeutic formulation 5 or the pressure sensitive adhesive 14 is pectin. The emulsifier (e.g., pectin) can be present in any suitable amount, provided the suitable amount is effective to emulsify the therapeutic formulation 5 or the pressure sensitive adhesive 14 and the emulsifier remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the emulsifier (e.g., pectin) can be present up to about 30.0 wt. % of the therapeutic formulation 5, in about 1.0 wt. % to about 20.0 wt. % of the therapeutic formulation 5, or in about 2.0 wt. % to about 10.0 wt. % of the therapeutic formulation 5.

Polymer

The pressure sensitive adhesive 14 can optionally include one or more polymers 9. The polymer 9 provides structure and strength to the pressure sensitive adhesive 14 or provides structure and strength to the therapeutic formulation 5. Any suitable polymer 9 can be employed, provided the polymer 9 provides structure and strength to the pressure sensitive adhesive 14 or provides structure and strength to the therapeutic formulation 5, and the polymer 9 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

Suitable polymers 9 include natural polymers and synthetic polymers. Specifically, the polymer 9 can include, e.g., karaya, a polyacrylamide, xanthum gum, guar gum, a hydrophilic polymer, a hydrocolloidal polymer, starch, a starch derivative, vinyl acetate copolymer, polyvinyl pyrrolidone, polyethylene oxide, algin, a derivative of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, or a combination thereof. Other suitable polymers 9 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably, the polymer 9 can include polyacrylamide, karaya, or a combination thereof.

Any suitable amount of polymer 9 can be employed, provided the amount of polymer 9 effectively provides structure and strength to the pressure sensitive adhesive 14 or to the therapeutic formulation 5, and the effective amount of polymer 9 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of polymer 9 will depend upon the specific polymer 9 employed. Specifically, karaya can be employed as the polymer 9 up to about 60 wt. % of the therapeutic formulation 5, in about 5.0 wt. % to about 45 wt. % of the therapeutic formulation 5, or in about 8.0 wt. % to about 40 wt. % of the therapeutic formulation 5; polyacrylamide can be employed as the polymer 9 up to about 40 wt. % of the therapeutic formulation 5, in about 5.0 wt. % to about 35 wt. % of the therapeutic formulation 5, or in about 8.0 wt. % to about 30 wt. % of the therapeutic formulation 5; or both karaya and polyacrylamide can be employed as the polymer 9, wherein karaya is present in about 5.0 wt. % to about 35 wt. % of the therapeutic formulation 5 and polyacrylamide is present in about 5.0 wt. % to about 30 wt. % of the therapeutic formulation 5.

Humectant

The therapeutic formulation 5 can optionally include one or more humectants 17 to provide a moistening effect to the pressure sensitive adhesive 14. The humectant 17 can optionally hydrate the polymer 9. Any suitable humectant 17 can be employed, provided the humectant 17 effectively provides a moistening effect to the pressure sensitive adhesive 14 and the humectant 17 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. One suitable humectant 17 is glycerin. Other suitable humectants 17 include polyhydric alcohols such as ethylene glycol, propylene glycol, triethylene glycol, tetraethylene glycol, sorbitol, and combinations thereof.

Any suitable amount of humectant 17 can be employed, provided the amount of humectant 17 effectively provides a moistening effect to the pressure sensitive adhesive 14 and the amount of humectant 17 effectively remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of humectant 17 will depend upon the specific humectant 17 employed and the specific polymer 9 employed. For example, karaya, polyacrylamide, or a combination thereof can be employed as the polymer 9 and glycerin can be employed as the humectant 17, wherein the glycerin is present in about 25.0 wt. % to about 70.0 wt. % or in about 40.0 wt. % to about 55.0 wt. % of the therapeutic formulation 5.

Filler

The therapeutic formulation 5 can optionally include one or more fillers 6. Any suitable filler 6 can be employed. Suitable fillers 6 include malto dextrin, dextrin, 70% sorbitol water, modified starches, depolymerized starches, and methylcellulose. As used herein, “malto dextrin” is a dextrose equivalent, wherein dextrose is D-glucose. Malto dextrin is commercially available as Amaizo Lodex 5 from American Maize-Products (Hammond, Ind.). Any suitable amount of filler can be employed in the therapeutic formulation 5. The suitable amount of filler can depend in part upon the specific filler present in the therapeutic formulation 5. For example, malto dextrin can be present up to about 20.0 wt. % of the therapeutic formulation 5, or in about 1.0 wt. % to about 10.0 wt. % of the therapeutic formulation 5.

Skin Protectant or Skin Conditioner

The therapeutic formulation 5 can optionally include a skin protectant 18 (i.e., topical moisturizer or skin conditioner). Any suitable skin protectant 18 can be employed, provided the skin is effectively protected or moisturized and the skin protectant remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the skin conditioner is pharmaceutically acceptable for topical use in humans.

Suitable topical moisturizers 18 include, e.g. calamine, aloe, lanolin, glycerin, Vitamin E, Vitamin E acetate, farnesol, glycyrrhetinic acid, aluminum hydroxide gel, cocoa butter, propylene glycol, ethylene glycol, triethylene glycol, hard fat, kaolin, mineral oil, petrolatum, topical starch, white petroleum, cod liver oil, shark liver oil, zinc oxide, or any combination thereof. Additional suitable topical moisturizers 18 are disclosed, e.g., in U.S. Pat. Nos. 6,096,334; 6,096,033; 5,741,510; 5,536,263; 4,675,009; 4,307,717; 4,274,420; 5,976,565; 5,536,263; and references cited therein.

As used herein, “aluminum hydroxide gel” refers to a suspension containing aluminum oxide (Al₂O₃), mainly in the form of a hydroxide. It is typically obtained by drying the product of interaction in aqueous solution of an aluminum salt with ammonium or sodium carbonate.

As used herein, “cocoa butter” refers to a fatty substance in cocoa beans; a thick oily solid obtained from cocoa beans and used in making chocolate, cosmetics, and suntan oil. Also known as threobroma oil, it lubricates and softens the skin.

As used herein, “topical starch” refers to cornstarch.

As used herein, “kaolin” refers to aluminum silicate; powdered and freed from gritty particles by elutriation. Kaolin refers to the name of the locality in China where the substance is found in abundance.

As used herein, “white petroleum” refers to a purified mixture of hydrocarbons obtained from petroleum. A bleached version of yellow soft paraffin, it is used as an emollient and as a base for ointments. It is odorless when rubbed into the skin and not readily absorbed.

As used herein, “mineral oil” refers to the heavy liquid petrolatum; liquid paraffin or petroleum; a mixture of liquid hydrocarbons obtained from petroleum, and is typically used as a vehicle in pharmaceutical preparations.

As used herein, “petrolatum” refers to petroleum jelly; a yellow soft paraffin; a yellowish mixture of the softer members of the paraffin or methane series of hydrocarbons, obtained from petroleum as an intermediate product in the distillation; typically used as a soothing application to burns and abrasions of the skin, and as a base for ointments.

As used herein, “cod liver oil” refers to the partially destearinated fixed oil extracted from the fresh livers of Gadus morrhuae and other species of the family Gadidae, containing Vitamins A and D.

As used herein, “shark liver oil” refers to the oil extracted from the livers of sharks, mainly of the species Hypoprion brevirostris; a rich source of Vitamins A and D.

As used herein, “zinc oxide” refers to ZnO, which is typically used as a protective ointment.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea. Aloe is commercially available as Aloe Vera Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

As used herein, “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; “lanolin” is the fat-like secretion of the sebaceous glands of sheep (i.e., complex mixture of esters and polyesters of 33 high molecular weight alcohols and 36 fatty acids) which is deposited onto the wool fibers; “famesol” is 3,7,11-trimethyl-2,6,10-dodecatrien-1-ol. Farnesol is commercially available from American Radiolabeled Chemicals (ARC) (St. Louis, Mo.), and “glycyrrhetinic acid” is a pentacyclic triterpenoid derivative of the beta-amyrin type and is shown below:

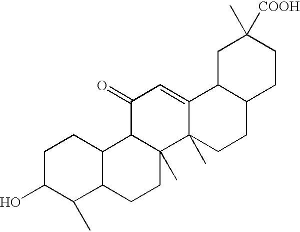

Any suitable amount of skin protectant 18 can be employed, provided the suitable amount of skin protectant 18 effectively protects or moisturizes the skin and the effective amount of skin protectant 18 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the amount of skin conditioner employed is pharmaceutically acceptable for topical use in humans.

Specifically, the skin protectant 18 can be present up to about 20.0 wt. %, up to 10.0 wt. %, up to 5.0 wt. %, or up to 2.0 wt. % of the therapeutic formulation 5. The suitable and effective amount of skin protectant 18 will depend in part upon the specific skin protectant 18 present in the therapeutic formulation 5. For example, Aloe Vera Gel, 10× can be present up to about 20.0 wt. % of the therapeutic formulation 5, up to about 10.0 wt. % of the therapeutic formulation 5, up to about 5.0 wt. % of the therapeutic formulation 5, or up to about 1.0 wt. % of the therapeutic formulation 5. In addition, Vitamin E acetate can be present up to about 10.0 wt. % of the therapeutic formulation 5, up to about 5.0 wt. % of the therapeutic formulation 5, up to about 3.0 wt. % of the therapeutic formulation 5, up to about 2.0 wt. % of the therapeutic formulation 5, or up to about 1.0 wt. % of the therapeutic formulation 5. Preferably, the skin conditioner will be present in an amount that is consistent with any State or Federal regulations, e.g., 21 C.F.R. Chapter 1, Part 346—Anorectal Drug Products for Over-The-Counter Human Use (§346.14).

The skin protectant 18 can be located in at least a portion of the front side 3 of the backing 2, on at least a portion of the front side 3 of the backing 2, or on and in at least a portion of the front side 3 of the backing 2. As such, the skin protectant 18 can be located on the entire surface of the front side 3 of the backing 2 or the skin protectant 18 can be located on a portion of the surface of the front side 3 of the backing 2. Preferably, the skin protectant 18 can be located on the entire surface of the front side 3 of the backing 2. In addition to being located on the surface of the front side 3 of the backing 2, the skin protectant 18 can be located in at least a portion of the underlying surface of the front side 3 of the backing 2 (i.e., the skin protectant 18 can be partially embedded into the backing 2). As shown in FIG. 9, the skin protectant 18 can penetrate a substantial portion of the front side 3 of the backing 2, as disclosed, e.g., in U.S. Pat. No. 5,536,263, and references cited therein. For example, the skin protectant 18 can penetrate about one-tenth to about nine-tenths the thickness of the backing 2, or about one-fourth to about nine-tenths the thickness of the backing 2. As such, the skin protectant 18 can be partially embedded into the backing 2. Preferably, the skin protectant 18 can be located on the entire front side 3 of the backing 2 and partially in the front side 3 of the backing 2 (i.e., the skin protectant 18 is partially embedded into the backing 2). Alternatively, a portion of the front side 3 of the backing 2 can include the skin protectant 18 and other portions of the front side 3 of the backing 2 can include any combination of the solvent 13, pressure sensitive adhesive 14, and vasoconstrictor 15. When the adhesive skin patch 1 is placed upon the skin of a patient (e.g., human), the skin protectant 18 can be in continuous contact with the skin surface of the patient.

Analgesic, Anesthetic and Antipruritic

The therapeutic formulation 5 can optionally include an analgesic, an anesthetic, an antipruritic, or a combination thereof. As used herein, an “antipruritic” refers to a substance that helps relieve or prevent itching; an “analgesic” refers to a substance that relieves pain; and an “anesthetic” refers to a substance that is capable of producing a complete or partial loss of feeling. Mosby's Medical Encyclopedia, CD-Rom version 2.0 (1997); and Mosby's Medical Nursing, & Allied Health Dictionary, Kenneth Anderson, 5th Ed., St. Louis, Mo. (1998). The analgesic is a topically applied substance that relieves pain by depressing cutaneous sensory receptors. 21 C.F.R. Chapter 1, Part 346—Anorectal Drug Products for Over-The-Counter Human Use. The anesthetic is a topically applied substance that relieves pain by depressing cutaneous sensory receptors. 21 C.F.R. Chapter 1, Part 346—Anorectal Drug Products for Over-The-Counter Human Use. The antipruritic is a topically applied substance that relieves itching by depressing cutaneous sensory receptors. 21 C.F.R. Chapter 1, Part 346—Anorectal Drug Products for Over-The-Counter Human Use.

Any suitable analgesic can be employed provided the analgesic effectively relieves the pain associated with hemorrhoidal tissue; any suitable anesthetic can be employed provided the anesthetic effectively produces a complete or partial loss of feeling to the topical area of the anus that is inflicted with the hemorrhoids; and any suitable antipruritic can be employed provided the antipruritic effectively relieves or prevents the itching associated with the hemorrhoids.

In one embodiment of the present invention, the analgesic, the anesthetic, the antipruritic, or the combination thereof can include camphor, menthol, benzocaine, butamben picrate, dibucaine, dimethisoquin, dyclonine, lidocaine, pramoxine, tetracaine, benzyl alcohol, camphorated metacresol, juniper tar, phenol, resorcinol, diphenhydramine, tripelennamine, hydrocortisone, hydrocortisone acetate, a pharmaceutically acceptable salt thereof, or a combination thereof.

In another embodiment of the present invention, the analgesic, the anesthetic, the antipruritic, or the combination thereof can include camphor, menthol, benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphorated metacresol, juniper tar, phenol, phenolate sodium, resorcinol, diphenhydramine hydrochloride, tripelennamine hydrochloride, hydrocortisone, hydrocortisone acetate, or a combination thereof.

The analgesic, anesthetic, antipruritic, or combination thereof can be present in any suitable and effective amount, provided the suitable amount of analgesic effectively relieves the pain associated with hemorrhoidal tissue; the suitable amount of anesthetic effectively produces a complete or partial loss of feeling to the topical area of the anus that is inflicted with the hemorrhoids; and the suitable amount of antipruritic effectively relieves or prevents the itching associated with the hemorrhoids. Preferably, the suitable amount of analgesic, anesthetic, antipruritic, or combination thereof is pharmaceutically acceptable for topical use in humans.

In one embodiment of the present invention, the camphor can be present up to about 3.0 wt. % of the therapeutic formulation 5 and menthol can be present up to about 1.0 wt. % of the therapeutic formulation 5; benzocaine can be present up to about 20.0 wt. % of the therapeutic formulation 5; butamben picrate can be present up to about 1.5 wt. % of the therapeutic formulation 5; dibucaine can be present up to about 1.0 wt. % of the therapeutic formulation 5; dibucaine hydrochloride can be present up to about 1.0 wt. % of the therapeutic formulation 5; dimethisoquin hydrochloride can be present up to about 0.5 wt. % of the therapeutic formulation 5; dyclonine hydrochloride can be present up to about 1.0 wt. % of the therapeutic formulation 5; lidocaine can be present up to about 4.0 wt. % of the therapeutic formulation 5; lidocaine hydrochloride can be present up to about 4.0 wt. % of the therapeutic formulation 5; pramoxine hydrochloride can be present up to about 1.0 wt. % of the therapeutic formulation 5; tetracaine can be present up to about 2.0 wt. % of the therapeutic formulation 5; tetracaine hydrochloride can be present up to about 2.0 wt. % of the therapeutic formulation 5; benzyl alcohol can be present up to about 33.0 wt. % of the therapeutic formulation 5; camphor can be present up to about 3.0 wt. % of the therapeutic formulation 5; juniper tar can be present up to about 5.0 wt. % of the therapeutic formulation 5; phenolate sodium can be present up to about 1.5 wt. % of the therapeutic formulation 5; resorcinol can be present up to about 3.0 wt. % of the therapeutic formulation 5; diphenhydramine hydrochloride can be present up to about 2.0 wt. % of the therapeutic formulation 5; tripelennamine hydrochloride can be present up to about 2.0 wt. % of the therapeutic formulation 5; hydrocortisone can be present up to about 1.0 wt. % of the therapeutic formulation 5; corticosteroid can be present up to about 5.0 wt. % of the therapeutic formulation 5; camphor can be present up to about 10.8 wt. % of the therapeutic formulation 5 with phenol; camphor can be present up to about 10.8 wt. % of the therapeutic formulation 5 with metacresol in about 1 wt. % to about 3.6 wt. % of the therapeutic formulation 5, as camphorated metacresol; and/or hydrocortisone acetate can be present up to about 1.0 wt. % of the therapeutic formulation 5.

In another embodiment of the present invention, camphor can be present up to about 3.0 wt. % of the therapeutic formulation 5 and menthol can be present up to about 1.0 wt. % of the therapeutic formulation 5; benzocaine can be present in about 5.0 wt. % to about 20.0 wt. % of the therapeutic formulation 5; butamben picrate can be present in about 0.5 wt. % to about 1.5 wt. % of the therapeutic formulation 5; dibucaine can be present in about 0.25 wt. % to about 1.0 wt. % of the therapeutic formulation 5; dibucaine hydrochloride can be present in about 0.25 wt. % to about 1.0 wt. % of the therapeutic formulation 5; dimethisoquin hydrochloride can be present in about 0.3 wt. % to about 0.5 wt. % of the therapeutic formulation 5; dyclonine hydrochloride can be present in about 0.5 wt. % to about 1.0 wt. % of the therapeutic formulation 5; lidocaine can be present in about 0.5 wt. % to about 4.0 wt. % of the therapeutic formulation 5; lidocaine hydrochloride can be present in about 0.5 wt. % to about 4.0 wt. % of the therapeutic formulation 5; pramoxine hydrochloride can be present in about 0.5 wt. % to about 1.0 wt. % of the therapeutic formulation 5; tetracaine can be present in about 1.0 wt. % to about 2.0 wt. % of the therapeutic formulation 5; tetracaine hydrochloride can be present in about 1.0 wt. % to about 2.0 wt. % of the therapeutic formulation 5; benzyl alcohol can be present in about 10.0 wt. % to about 33.0 wt. % of the therapeutic formulation 5; camphor can be present in about 0.1 wt. % to about 3.0 wt. % of the therapeutic formulation 5; juniper tar can be present in about 1.0 wt. % to about 5.0 wt. % of the therapeutic formulation 5; phenolate sodium can be present in about 0.5 wt. % to about 1.5 wt. % of the therapeutic formulation 5; resorcinol can be present in about 0.5 wt. % to about 3.0 wt. % of the therapeutic formulation 5; diphenhydramine hydrochloride can be present in about 1.0 wt. % to about 2.0 wt. % of the therapeutic formulation 5; tripelennamine hydrochloride can be present in about 0.5 wt. % to about 2.0 wt. % of the therapeutic formulation 5; hydrocortisone can be present in about 0.25 wt. % to about 1.0 wt. % of the therapeutic formulation 5; corticosteroid can be present in about 0.25 to about 5.0 wt. % of the therapeutic formulation 5; camphor can be present in about 3 wt. % to about 10.8 wt. % of the therapeutic formulation 5 with phenol; camphor can be present in about 3 wt. % to about 10.8 wt. % of the therapeutic formulation 5 with metacresol in about 1 wt. % to about 3.6 wt. % of the therapeutic formulation 5, as camphorated metacresol; and/or hydrocortisone acetate can be present in about 0.25 wt. % to about 1.0 wt. % of the therapeutic formulation 5.

In another embodiment of the present invention, hydrocortisone, hydrocortisone acetate, or a combination thereof can be present in about 0.25 wt. % to about 1.0 wt. % of the therapeutic formulation 5; lidocaine, lidocaine hydrochloride, or a combination thereof can be present in about 0.5 wt. % to about 4.0 wt. % of the therapeutic formulation 5; camphor can be present in about 0.1 wt. % of the therapeutic formulation 5 to about 3.0 wt. % of the therapeutic formulation 5; juniper tar can be present in about 1.0 wt. % of the therapeutic formulation 5 to about 5.0 wt. % of the therapeutic formulation 5; and/or menthol can be present in about 0.1 wt. % of the therapeutic formulation 5 to about 1.0 wt. % of the therapeutic formulation 5.

In another embodiment of the present invention, benzocaine can be present in about 5.0 wt. % of the therapeutic formulation 5 to about 20.0 wt. % of the therapeutic formulation 5; benzyl alcohol can be present in about 1.0 wt. % of the therapeutic formulation 5 to about 4.0 wt. % of the therapeutic formulation 5; dibucaine can be present in about 0.25 wt. % of the therapeutic formulation 5 to about 1.0 wt. % of the therapeutic formulation 5; dibucaine hydrochloride can be present in about 0.25 wt. % of the therapeutic formulation 5 to about 1.0 wt. % of the therapeutic formulation 5; dyclonine hydrochloride can be present in about 0.5 wt. % of the therapeutic formulation 5 to about 1.0 wt. % of the therapeutic formulation 5; lidocaine can be present in about 2.0 wt. % of the therapeutic formulation 5 to about 5.0 wt. % of the therapeutic formulation 5; pramoxine hydrochloride can be present in about 0.5 wt. % of the therapeutic formulation 5 to about 1.5 wt. % of the therapeutic formulation 5; tetracaine can be present in about 0.5 wt. % of the therapeutic formulation 5 to about 1.0 wt. % of the therapeutic formulation 5; and/or tetracaine hydrochloride can be present in about 0.5 wt. % of the therapeutic formulation 5 to about 1.0 wt. % of the therapeutic formulation 5.

The analgesic, anesthetic, antipruritic, or combination thereof can be located in at least a portion of the front side 3 of the backing 2, on at least a portion of the front side 3 of the backing 2, or on and in at least a portion of the front side 3 of the backing 2. As such, the analgesic, anesthetic, antipruritic, or combination thereof can be located on the entire surface of the front side 3 of the backing 2 or the analgesic, anesthetic, antipruritic, or combination thereof can be located on a portion of the surface of the front side 3 of the backing 2. Preferably, the analgesic, anesthetic, antipruritic, or combination thereof can be located on the entire surface of the front side 3 of the backing 2. In addition to being located on the surface of the front side 3 of the backing 2, the analgesic, anesthetic, antipruritic, or combination thereof can be located in at least a portion of the underlying surface of the front side 3 of the backing 2 (i.e., the analgesic, anesthetic, antipruritic, or combination thereof can be partially embedded into the backing 2). As shown in FIG. 9, the analgesic, anesthetic, antipruritic, or combination thereof can penetrate a substantial portion of the front side 3 of the backing 2, as disclosed, e.g., in U.S. Pat. No. 5,536,263, and references cited therein. For example, the analgesic, anesthetic, antipruritic, or combination thereof can penetrate about one-tenth to about nine-tenths the thickness of the backing 2, or about one-fourth to about nine-tenths the thickness of the backing 2. As such, the analgesic, anesthetic, antipruritic, or combination thereof can be partially embedded into the backing 2. Preferably, the analgesic, anesthetic, antipruritic, or combination thereof can be located on the entire front side 3 of the backing 2 and partially in the front side 3 of the backing 2 (i.e., the analgesic, anesthetic, antipruritic, or combination thereof is partially embedded into the backing 2). Alternatively, a portion of the front side 3 of the backing 2 can include the analgesic, anesthetic, antipruritic, or combination thereof and other portions of the front side 3 of the backing 2 can include any combination of the solvent 13 and vasoconstrictor 15. When the adhesive skin patch 1 is placed upon the skin of a patient (e.g., human), the analgesic, anesthetic, antipruritic, or combination thereof can be in continuous contact with the skin surface of the patient.

Anti-Inflammatory Agent

The therapeutic formulation 5 can optionally include an anti-inflammatory agent. As used herein, an “anti-inflammatory agent” refers to a substance that reduces inflammation or swelling of hemorrhoidal tissue. Mosby's Medical Encyclopedia, CD-Rom version 2.0 (1997); and Mosby's Medical, Nursing, & Allied Health Dictionary, Kenneth Anderson, 5th Ed., St. Louis, Mo. (1998). Any suitable anti-inflammatory agent can be employed, provided the anti-inflammatory agent effectively reduces inflammation or swelling of hemorrhoidal tissue.

In one embodiment of the present invention, the suitable anti-inflammatory agent can be a corticosteroid. As used herein, a “corticosteroid” refers to any one of the natural or synthetic hormones elaborated by the adrenal gland (adrenal cortex). Mosby's Medical Encyclopedia, CD-Rom version 2.0 (1997); and Mosby's Medical, Nursing, & Allied Health Dictionary, Kenneth Anderson, 5th Ed., St. Louis, Mo. (1998). Any suitable corticosteroid can be employed, provided the corticosteroid reduces inflammation or swelling of hemorrhoidal tissue. Preferably, the suitable corticosteroid is pharmaceutically acceptable for topical use in humans. Suitable corticosteroids are known to those of skill in the art and are disclosed, e.g., in Goodman Gilman, Alfred; Goodman, Louis S.; Gilman, Alfred; Goodman and Gilman's The Pharmacological Basis of Therapeutics, Sixth Edition, pp.1482-1486; and Christophers, Enno; Schöpf, Erwin; Kligman, Albert M.; Stoughton, Richard B.; Topical Corticosteroid Therapy; A Novel Approach to Safer Drugs, Raven Press, pp. 3-5.

Suitable exemplary corticosteroids include cortisol (hydrocortisone); tetrahydrocortisol; prednisone (cortisone); prednisolone (cortisol); 6α-methylprednisolone; fludrocortisone (9α-fluorocortisol); 11-desoxycortisol; cortisone (11-dehydrocortisol); corticosterone; triamcinolone (9α-fluoro-16α-hydroxyprednisolone); paramethasone (6α-fluoro-16α-methylprednisolone); betamethasone (9α-fluoro-16β-methylprednisolone); dexamethasone (9α-fluoro-16α-methylprednisolone); desoxycorticosterone acetate (doca acetate, percorten acetate); desoxycorticosterone pivalate (percorten pivalate); fludrocortisone acetate (florine acetate); cortisol (hydrocortisone) (cortef, hydrocortone); cortisol acetate (cortef acetate, hydrocortone acetate); cortisol cypionate (cortef); cortisol sodium phosphate (hydrocortone phosphate); cortisol sodium succinate (solu-cortef); beclopmethasone dipropionate (vanceril); betamethasone (celestone); betamethasone sodium phosphate and acetate (celestone soluspan); betamethasone dipropionate (diprosone); betamethasone valerate (valisone); betamethasone benzoate (benisone, flurodate); cortisone acetate (cortone acetate); dexamethasone (decadron, gammacorten); dexamethasone sodium phosphate (decadron phosphate, hexadrol phosphate); dexamethasone acetate (decadron-L.A.); fuprednisolone (alphadrol); meprednisone (betapar); methylprednisolone (medrol); methylprednisolone acetate (depo-medrol, medrol acetate); methylprednisolone sodium succinate (solu-medrol); paramethasone acetate (haldrone); prednisolone (delta-cortef); prednisolone acetate (meticortelone acetate); prednisolone sodium phosphate (hydeltrasol); prednisolone sodium succinate (meticortelone soluble); prednisolone tebutate (hydelta-T.B.A.); prednisone (deltasone, paracort); triamcinolone (aristocort, kenacort); triamcinolone acetonide (aristoderm, kenalog); triamcinolone diacetate (aristocort diacetate, kienacort diacetate); triamcinolone hexacotonide (aristospan); desonide (tridesilon); desoximetasone (topicort); flumethasone pivalate (locorten); fluocinolone acetonide (fluonid, synalar); fluocinonide (lidex, topsyn); fluorometholone (oxylone); flurandrenolide (cordran); halcinonide (halog); and medrysone (HMS liquifilm, medrocort). See, e.g., Goodman Gilman, Alfred; Goodman, Louis S.; Gilman, Alfred; Goodman and Gilman's The Pharmacological Basis of Therapeutics, Sixth Edition, pp.1482-1486 (Tables 63-3 and 63-4).

Additional suitable exemplary corticosteroids include aclometasone dipropionate (alclovate); betamethasone-17-benzoate (benisone, flurobate); betamethasone dipropionate (diprosone); betamethasone-17-valerate (valisone); clobetasol propionate (temovate); desonide (desowen, tridesilon); dexamethasone (aeroseb-D); desoximetasone (topicort); diflorasone diacetate (florone); flumethasone pivalate (locorten); fluocinolone acetonide (synalar, synalar-HP, neosynalar, fluonid); fluocinolone acetonide acetate (lidex; lidex-E; topsyn); fluorometholone (oxylone); flurandrenolide (cordran); halcinonide (halog); hydrocortisone (cort-dome, lubricort); hydrocortisone acetate (cortef, carmol HC, neo-cortef); hydrocortisone-17-valerate (westcort); prednisolone (meti-derm); and triamcinolone acetonide (kenalog, orabase, kenalog-S, mycolog, aristocort, aristocort-A, aristoderm, neo-aristoderm, neo-aristocort). See, e.g., Christophers, Enno; Schöpf, Erwin; Kligman, Albert M.; Stoughton, Richard B.; Topical Corticosteroid Therapy; A Novel Approach to Safer Drugs, Raven Press, pp. 3-4 (Table 1).

Additional suitable exemplary corticosteroids include temovate; diprolen; psorcon; temovate; diprolene; cyclocort; diprosone; florone; halog; lidex; maxiflor; topicort; aristocort A; diprosone; florone; maxiflor; valisone; cordran; kenalog; synalar; topicort LP; westcort; cordran; diprosone; kenalog; locold; synalar; valisone; westcort; aclovate; desowen; locorten; synalar; tridesilone; valisone; hydrocortisone; dexamethasone; flumethalone; prednisolone; and methylprednisolone. See, e.g., Christophers, Enno; Schöpf, Erwin; Kligman, Albert M.; Stoughton, Richard B.; Topical Corticosteroid Therapy; A Novel Approach to Safer Drugs, Raven Press, p. 5 (Table 2).

Additional suitable exemplary corticosteroids include diprolene and diprosone. See, e.g., Christophers, Enno; Schöpf, Erwin; Kligman, Albert M.; Stoughton, Richard B.; Topical Corticosteroid Therapy; A Novel Approach to Safer Drugs, Raven Press, p. 5 (Table 3).

Additional suitable exemplary corticosteroids include augmented betamethasone dipropionate (diprolene); diflorasone diacetate (psorcon); clobetasol propionate (temovate); halobetasol propionate (ultravate); amcinonide (cyclocort); betamethasone dipropionate (diprolene, diprosone); diflorasone diacetate (florone); halcinonide (halog); fluocinonide (lidex); diflorasone diacetate (maxiflor); betamethasone dipropionate (maxivate); diflorasone diacetate (psorcom); desoximetasone (topicort); (aristocort A); amcinonide (cyclocort); betamethasone dipropionate (diprosone); mometasone furoate (elocon); diflorasone diacetate (florone); halcinonide (halog); fluocinonide (lidex-E); diflorasone diacetate (maxiflor); betamethasone dipropionate (maxivate, psorion); betamethasone valerate (valisone); flurandrenolide (cordran); fluticasone propionate (cutivate); mometasone furoate (elocon); triamcinolone acetonide (kenalog); fluocinolone acetonide (synalar); hydrocortisone valerate (westcort); flurandrenolide (cordran); fluticasone propionate (cutivate); betamethasone dipropionate (diprosone); triamcinolone acetonide (kenalog); hydrocortisone butyrate (locoid); fluocinolone acetonide (synalar); betamethasone valerate (valisone); hydrocortisone valerate (westcort); alclometasone dipropionate (aclovate); triamcinolone acetonide (aristocort); desonide (desowen); flumethasone pivalate (locorten); fluocinolone acetonide (synalar); desonide (tridesilon); betamethasone valerate (valisone); hydrocortisone (eldecort, dexamethasone, flumethalone, hydrocortisone, methylprednisolone, or prednisolone); betamethasone; and dexamethasone.

The suitable corticosteroid can be present in any suitable amount, provided the amount of corticosteroid effectively treats or prevents a condition associated with hemorrhoids and the amount is stable in the therapeutic formulation 5 over a prolonged period of time typically experienced in the manufacturing, packaging, shipping, and/or the storage of the patch. Preferably, the amount of suitable corticosteroid is pharmaceutically acceptable for topical use in humans. The suitable corticosteroid can be present in about 0.1 wt. % to about 99.9 wt. % of the therapeutic formulation 5. Typically, the amount of corticosteroid present will depend upon the specific corticosteroid or corticosteroids employed in the therapeutic formulation 5. Specifically, the corticosteroid can be up to about 10 wt. %, up to about 5 wt. %, up to about 2 wt. %, up to about 1 wt. %, or up to about 0.1 wt. % of the therapeutic formulation 5. Additionally, the nature and amount of corticosteroid present in the therapeutic formulation 5 should comply with any State and/or Federal guidelines that regulate the use of such compounds (e.g., FDA regulations).

Antibiotic Agent

The therapeutic formulation 5 can optionally include one or more suitable antibiotic agents 16 (i.e., antimicrobial agent). The presence of the antibiotic agent will effectively treat or prevent a bacterial infection associated with hemorrhoids or will effectively treat or prevent a bacterial infection associated with a surgical procedure to treat hemorrhoids. As used herein, an “antibiotic agent” or “antimicrobial agent” is any compound having activity against either Gram-positive or Gram-negative organisms (i.e., inhibits the growth or destroys the development of either Gram-positive or Gram-negative organisms). Stedman's Medical Dictionary Illustrated, (25th Ed.), Williams & Wilkins: Baltimore (1990) and Mosby's Medical, Nursing & Allied Health Dictionary, (5th Ed.), Mosby: St. Louis (1998).

Any suitable antibiotic agent 16 can be employed, provided the antibiotic agent 16 effectively inhibits the growth or destroys the development of either Gram-positive or Gram-negative organisms and the antibiotic agent 16 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Suitable antibiotic agents 16 are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references cited therein.

Suitable classes of antibiotic agents 16 include, e.g., β-lactams, aminoglycosides, antifungal agents, and combinations thereof. Suitable antibiotic agents 16 include, e.g., cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole, sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, pinworm, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscamet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscamet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, pharmaceutically acceptable salts thereof, and combinations thereof. Specifically, the antibiotic agent can be erythromycin, tetracycline, clindamycin, cephalosporin, pharmaceutically acceptable salts thereof, or a combination thereof.

Any suitable amount of antibiotic agent 16 can be employed, provided the amount of antibiotic agent 16 employed effectively inhibits the growth or destroys the development of either Gram-positive or Gram-negative organisms and the effective amount of the antibiotic agent 16 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The antibiotic agent 16 can be present up to about 99.9 wt. % of the therapeutic formulation 5, up to about 50 wt. % of the therapeutic formulation 5, up to about 25 wt. % of the therapeutic formulation 5, or up to about 10 wt. % of the therapeutic formulation 5. Typically, the amount of antibiotic agent 16 will depend upon the specific antibiotic agent 16 employed. Preferably, the antibiotic agent 16 can be present up to about 5.0 wt. % of the therapeutic formulation 5, up to about 1.0 wt. % of the therapeutic formulation 5, or up to about 0.5 wt. % of the therapeutic formulation 5.

The antibiotic agent 16 can be located in at least a portion of the front side 3 of the backing 2, on at least a portion of the front side 3 of the backing 2, or on and in at least a portion of the front side 3 of the backing 2. As such, the antibiotic agent 16 can be located on the entire surface of the front side 3 of the backing 2 or the antibiotic agent 16 can be located on a portion of the surface of the front side 3 of the backing 2. Preferably, the antibiotic agent 16 can be located on the entire surface of the front side 3 of the backing 2. In addition to being located on the surface of the front side 3 of the backing 2, the antibiotic agent 16 can be located in at least a portion of the underlying surface of the front side 3 of the backing 2 (i.e., the antibiotic agent 16 can be partially embedded into the backing 2). As shown in FIG. 9, the antibiotic agent 16 can penetrate a substantial portion of the front side 3 of the backing 2, as disclosed, e.g., in U.S. Pat. No. 5,536,263, and references cited therein. For example, the antibiotic agent 16 can penetrate about one-tenth to about nine-tenths the thickness of the backing 2, or about one-fourth to about nine-tenths the thickness of the backing 2. As such, the antibiotic agent 16 can be partially embedded into the backing 2. Preferably, the antibiotic agent 16 can be located on the entire front side 3 of the backing 2 and partially in the front side 3 of the backing 2 (i.e., the antibiotic agent 16 is partially embedded into the backing 2). Alternatively, a portion of the front side 3 of the backing 2 can include the antibiotic agent 16 and other portions of the front side 3 of the backing 2 can include any combination of the solvent 13, pressure sensitive adhesive 14, and vasoconstrictor 15. When the adhesive skin patch 1 is placed upon the skin of a patient (e.g., human), the antibiotic agent 16 can be in continuous contact with the skin surface of the patient.

Antiseptic

The therapeutic formulation 5 can optionally include an antiseptic 30. As used herein, an “antiseptic” is an agent or substance capable of effecting antisepsis, i.e., the prevention of infection by inhibiting the growth of infectious agents. Stedman's Medical Dictionary, 25th Ed., illustrated, Williams & Wilkins, Baltimore, Md., p. 100 (1990). Any suitable antiseptic 30 can be employed, provided the suitable antiseptic 30 effectively inhibits the growth of infectious agents and the effective antiseptic 30 remains stable in the therapeutic formulation 5. Suitable antiseptics 30 include, e.g., triclosan, phenoxy isopropanol, chlorhexidine gluconate, povidone iodine, and any combination thereof.

The antiseptic 30 can be employed in any suitable amount, provided the suitable amount of antiseptic 30 effectively inhibits the growth of infectious agents and maintains the stability of the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. For example, the antiseptic 30 can be employed up to about 20.0 wt. % of the of the therapeutic formulation 5, up to about 10.0 wt. % of the of the therapeutic formulation 5, up to about 1.0 wt. % of the of the therapeutic formulation 5, or up to about 0.1 wt. % of the of the therapeutic formulation 5.

The antiseptic 30 can be located in at least a portion of the front side 3 of the backing 2, on at least a portion of the front side 3 of the backing 2, or on and in at least a portion of the front side 3 of the backing 2. As such, the antiseptic 30 can be located on the entire surface of the front side 3 of the backing 2 or the antiseptic 30 can be located on a portion of the surface of the front side 3 of the backing 2. Preferably, the antiseptic 30 can be located on the entire surface of the front side 3 of the backing 2. In addition to being located on the surface of the front side 3 of the backing 2, the antiseptic 30 can be located in at least a portion of the underlying surface of the front side 3 of the backing 2 (i.e., the antiseptic 30 can be partially embedded into the backing 2). As shown in FIG. 9, the antiseptic 30 can penetrate a substantial portion of the front side 3 of the backing 2, as disclosed, e.g., in U.S. Pat. No. 5,536,263, and references cited therein. For example, the antiseptic 30 can penetrate about one-tenth to about nine-tenths the thickness of the backing 2, or about one-fourth to about nine-tenths the thickness of the backing 2. As such, the antiseptic 30 can be partially embedded into the backing 2. Preferably, the antiseptic 30 can be located on the entire front side 3 of the backing 2 and partially in the front side 3 of the backing 2 (i.e., the antiseptic 30 is partially embedded into the backing 2). Alternatively, a portion of the front side 3 of the backing 2 can include the antiseptic 30 and other portions of the front side 3 of the backing 2 can include any combination of the solvent 13, pressure sensitive adhesive 14, and vasoconstrictor 15. When the adhesive skin patch 1 is placed upon the skin of a patient (e.g., human), the antiseptic 30 can be in continuous contact with the skin surface of the patient.

Preservative

The therapeutic formulation 5 can optionally include a preservative 7 that is useful for preventing bacterial growth, mold growth, fermentation, and/or decomposition. As used herein, “preservative” refers to any substance which prevents bacterial growth, mold growth, fermentation, and/or decomposition. Concise Chemical and Technical Dictionary, 4th enlarged edition, Chemical Publishing Co., Inc., NY, N.Y. p. 939 (1986). Any suitable preservative 7 can be employed, provided the preservative 7 effectively prevents bacterial growth, mold growth, fermentation, and/or decomposition; and the preservative 7 remains stable in the therapeutic formulation 5. Preferably, the stability is over a prolonged period of time, e.g., up to about 2 years, up to about 1 year, or up to about 6 months, typically experienced in the manufacturing, packaging, shipping, and/or storage of the adhesive skin patch 1.

Suitable preservatives 7 include, e.g., quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2 -thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and pharmaceutically acceptable salts thereof. Preferably, the preservative is quat-15, which is commercially available from Dow Chemical (Midland Mich.); methyl paraben; ascorbic acid; or a combination thereof.

The preservative 7 can be employed in any suitable amount provided the amount of preservative 7 effectively prevents bacterial growth, mold growth, fermentation, and/or decomposition and the effective amount of preservative 7 remains stable in the therapeutic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The preservative 7 can be present up to about 99.9 wt. % of the therapeutic formulation 5, up to about 20.0 wt. % of the therapeutic formulation 5, up to 5.0 wt. % of the therapeutic formulation 5, or up to 1.5 wt. % of the therapeutic formulation 5. The amount of preservative 7 present in the therapeutic formulation 5 will typically depend upon the specific compound or compounds employed as the preservative 7. For example, quat-15 can be employed in about 0.01 wt. % to about 1.5 wt. % of the therapeutic formulation 5, in about 0.05 wt. % to about 0.15 wt. % of the therapeutic formulation 5, or in about 0.08 wt. % to about 0.12 wt. % of the therapeutic formulation 5.

Complexing Agent

In one embodiment of the present invention, the therapeutic formulation 5 can include a component (e.g., anti-inflammatory agent) that is not soluble and/or stable in the solvent 13, in the amount employed. The use of a complexing agent can be employed to solubilize and/or stabilize these components in the therapeutic formulation 5. Any suitable complexing agent can be employed, provided the complexing agent effectively solubilizes and/or stabilizes these components and the complexing agent remains stable in the therapeutic formulation 5 over a prolonged period of time. Preferably, the stability is over a prolonged period of time, e.g., up to about 3 years, up to about 1 year, or up to about 6 months, typically experienced in the manufacturing, packaging, shipping, and/or storage of the adhesive skin patch 1. In addition, any suitable amount of complexing agent can be employed, provided the amount of complexing agent effectively solubilizes and/or stabilizes these components and the amount of complexing agent remains stable in the therapeutic formulation 5 over a prolonged period of time.

For example, at standard temperature and pressure, corticosteroids such as hydrocortisone are not typically soluble or stable in aqueous solutions. It has surprisingly been discovered, however, that a suitable complexing agent such as a cyclodextrin can be employed to solubilize and/or stabilize the corticosteroid in the aqueous solution. As used herein, a “cyclodextrin” refers to a non-reducing cyclic oligosaccharide with at least 6 anhydroglucose units linked by alpha 1,4 bonds to form a ring. Cyclodextrins are typically produced by the action of the enzyme cyclodextrin glucosyltransferase [CGT-ase] on starch. The most common cyclodextrins include alpha, beta, and gamma cyclodextrins, which have six, seven, or eight, respectively, anhydroglucose units in the ring structure. All of the hydroxyl groups in cyclodextrins are oriented to the outside of the ring while the glucosidic oxygen and two rings of the non-exchangeable hydrogen atoms are directed towards the interior of the cavity. This combination gives cyclodextrins a hydrophobic inner cavity and a hydrophilic exterior. See, e.g., the Cerestar website (http://www.cerestar.com); the Betadexcyclodextrin website (http://www.betadexcyclodextrin.com); and M. L. Bender and M. Komiyama, Cyclodextrin Chemistry, Springer, Berlin, 1978.

Cyclodextrins are enzymatically-modified starches formed by the action of the enzyme cyclodextrin glucosyltransferase on starch. They are doughnut-shaped molecules, which can interact with organic molecules to form complexes. It is also possible for some organic molecules and some inorganic salts to associate with the hydroxyl groups of the cyclodextrin. Three cyclodextrins are typically formed, alpha, beta, and gamma cyclodextrin, which contain six, seven, or eight glucose molecules in the ring, respectively. The electron-dense glycosidic oxygen atoms are oriented inward and line the cavity. The hydroxyl groups are directed toward the outside of the ring. These hydrophilic groups interact with the water to give the cyclodextrins their aqueous solubility properties. The hydrogen and glycosidic oxygen atoms lining the cavity give the cyclodextrin molecule its hydrophobic character and its ability to interact with organic molecules to form complexes. Because of the free rotation of the C-6 carbon, this end of the cyclodextrin cavity is narrower than the end with the C-2 and C-3 hydroxyls.

Derivatives of cyclodextrin can be obtained, e.g., by replacing one or more hydroxyl groups with a suitable radical (i.e., pendant group). Suitable pendant groups include, e.g., sulfinyl; sulfonyl; phosphate; (C₁-C₁₂)alkyl optionally substituted with one or more (e.g., 1, 2, 3, or 4) hydroxy, carboxy, carbonyl, acyl, oxy, oxo; or a combination thereof. Suitable specific pendant groups include methyl, ethyl, hydroxypropyl, carboxymethyl, sulfate, phosphate, and an acrylate. For example, the specific pendant group can include (C₁-C₁₂)alkoxy optionally substituted with one or more hydroxy.

Specific suitable derivatives of cyclodextrin include, e.g., alpha-cyclodextrin sulfate, beta-cyclodextrin sulfate, gamma-cyclodextrin sulfate, alpha-hydroxypropyl cyclodextrin, beta-hydroxypropyl cyclodextrin, gamma-hydroxypropyl cyclodextrin, alpha-cyclodextrin phosphate, beta-cyclodextrin phosphate, and gamma-cyclodextrin phosphate.

Cyclodextrins are starches that have been specially modified by the action of an enzyme to make a water-soluble ring-shaped molecule, capable of holding another, oil-like organic substance in its ‘cavity’. Because of this unique property, cyclodextrins can be used to carry all kinds of active ingredients (e.g., drugs, fragrances, flavors, and vitamins) in a wide variety of formulations. Increased stability, water solubility, and controlled release are among the many application benefits. Specifically, cyclodextrins have the benefit of encapsulating a substance, thereby providing protection for the substance. This results in increased shelf-life and a reduced loss of degradation or decomposition. Cyclodextrins are themselves soluble in water, and can greatly increase the solubility of highly water insoluble substances. In addition, cyclodextrins can be used to control the release of a substance.

Suitable cyclodextrins include alpha cyclodextrins, beta cyclodextrins, and gamma cyclodextrins. Specifically, the cyclodextrin can be hydroxylpropyl beta cyclodextrin, hydroxylproplyl alpha cyclodextrin, or a combination thereof. In addition, the cyclodextrin can optionally be branched.

Suitable cyclodextrins, and derivatives thereof, can be found, e.g., at U.S. Pat. No. 5,376,641; U.S. Pat. No. 5,229,370; U.S. Pat. No. 4,383,992; the Cerestar website (http://www.cerestar.com); the Betadexcyclodextrin website (http://www.betadexcyclodextrin.com); French et al., Archives in Biochem. and Biophysics, Volume III, (1965) 153-150; the carbomer website (http://www.carbomer.com) and references cited therein.

Astringent

The therapeutic formulation 5 can optionally include an astringent. As used herein, an “astringent” refers to a substance that causes tissue (e.g., a hemorrhoidal) to contract and can optionally arrest secretion or control bleeding from tissue. Mosby's Medical Encyclopedia, CD-Rom version 2.0 (1997); and Mosby's Medical Nursing, & Allied Health Dictionary, Kenneth Anderson, 5th Ed., St. Louis, Mo. (1998). The astringent is a substance that is applied topically to the skin or mucuous membranes for local and limited protein coagulant effect. 21 C.F.R. Chapter 1, Part 346—Anorectal Drug Products for Over-The-Counter Human Use. Any suitable astringent can be employed, provided the astringent effectively causes hemorrhoidal tissue to contract. Preferably, the astringent is pharmaceutically acceptable for topical use in humans. Suitable astringents include, e.g., alum, tannic acid, calamine, witch hazel, zinc oxide, or a combination thereof.

As used herein, “with hazel” refers to hamamelis; “alum” refers to a double sulfate salt of aluminum and a monovalent metal that is typically used as an astringent in lotions and douches; and “tannic acid” refers to a group of compounds (tannins) with astringent taste obtained from the bark, fruits, and leaves of many plants (e.g., bark of oak).

Any suitable and effective amount of astringent can be employed, provided the astringent effectively causes hemorrhoidal tissue to contract. The amount of astringent employed can be up to about 40 wt. % of the of the therapeutic formulation 5, or up to about 25 wt. % of the thetapeutic formulation 5. The amount of astringent employed will typically depend upon the specific astringent or astringents employed. Specifically, calamine can be present up to about 25 wt. % of the therapeutic formulation 5; witch hazel can be present up to about 50 wt. % of the therapeutic formulation 5; and/or zinc oxide can be present up to about 25 wt. % of the therapeutic formulation 5. More specifically, calamine can be present in about 5 wt. % to about 25 wt. % of the therapeutic formulation 5; witch hazel can be present in about 10 wt. % to about 50 wt. % of the therapeutic formulation 5; and/or zinc oxide can be present in about 5 wt. % to about 25 wt. % of the therapeutic formulation 5.

Keratolytic Agent

The therapeutic formulation 5 can optionally include a keratolytic agent. As used herein, a “keratolytic agent” refers to a substance that causes desquamation (loosening) and debridement or sloughing of the surface cells of the epidermis. 21 C.F.R. Chapter 1, Part 346—Anorectal Drug Products for Over-The-Counter Human Use. Any suitable keratolytic agent can be employed, provided the keratolytic agent effectively causes desquamation (loosening) and debridement or sloughing of the surface cells of the epidermis of the region of the anus. Preferably, the keratolytic agent is pharmaceutically acceptable for topical use in humans.

Suitable keratolytic agents include, e.g., alcloxa, resorcinol, or a combination thereof. As used herein, “alcloxa” refers to Al-chlorhydroxy allontoinate; and “resorcinol” refers to m-dihydroxybenzene or 1,3-benzenediol.

Any suitable and effective amount of keratolytic agent can be employed, provided amount of keratolytic agent effectively causes desquamation (loosening) and debridement or sloughing of the surface cells of the epidermis of the region of the anus. Preferably, the amount of keratolytic agent is pharmaceutically acceptable for topical use in humans. The amount of keratolytic agent will typically depend upon the specific keratolytic agent or keratolytic agents employed. Specifically, alcloxa can be present up to about 2.0 wt. % of the therapeutic formulation 5; and/or resorcinol can be present up to about 3.0 wt. % of the thetapeutic formulation 5. More specifically, alcloxa can be present in about 0.2 wt. % to about 2.0 wt. % of the therapeutic formulation 5; and/or resorcinol can be present in about 1.0 wt. % to about 3.0 wt. % of the thetapeutic formulation 5.

The therapeutic formulation 5 can preferably remain stable over the period of time typically experienced with the manufacturing, packaging, shipping, and/or storage of the adhesive skin patch 1, e.g., up to about a month, up to about a year, up to about two years, or up to about 3 years. The stability of the vasoconstrictor 15, for example, is due in part to the therapeutic formulation 5 including the vasoconstrictor 15 in an adhesive formulation. The adhesive formulation is preferably a hydrogel that holds the vasoconstrictor 15 in an available form while maintaining the necessary stability, pressure sensitive adhesion and effectiveness over a prolonged period of time, e.g., up to about a month, up to about a year, up to about two years, or up to about 3 years.

The adhesive skin patch 1 can have any suitable size and shape. In addition, the adhesive skin patch 1 can be cut, as desired, to provide an adhesive skin patch 1 of a desired size and shape. The adhesive skin patch 1 can be cut with any suitable cutting device such as a scissors, scalpel, or knife.

The adhesive skin patch 1 can have any suitable length. In one embodiment of the present invention, the patch can be a self-wound roll 25 without a release liner 10 mounted on the front side 3 of the backing 2 of the adhesive skin patch 1. See, e.g., FIG. 10. In such an embodiment, the adhesive skin patch 1 can have a length of about 12 inches to about 100 yards, about 10 feet to about 50 yards, or about 20 feet to about 20 yards.

In one embodiment of the present invention, the adhesive skin patch 1 can be rectangular and can have a release liner 10 mounted on the front side 3 of the backing 2 of the adhesive skin patch 1. In such an embodiment, the adhesive skin patch 1 can typically have a length of up to about 10 inches, up to about 8 inches, up to about 5 inches, or up to about 3 inches. The adhesive skin patch 1 can have any suitable width. Typically, the adhesive skin patch 1 will have a width of up to about 10 inches, up to about 8 inches, up to about 5 inches, or up to about 3 inches. Additionally, the adhesive skin patch 1 can have any suitable thickness. Typically, the adhesive skin patch 1 will have a thickness of about 0.10 mm to about 2.0 mm, about 0.15 mm to about 1.0 mm, or about 0.20 mm to about 0.75 mm.

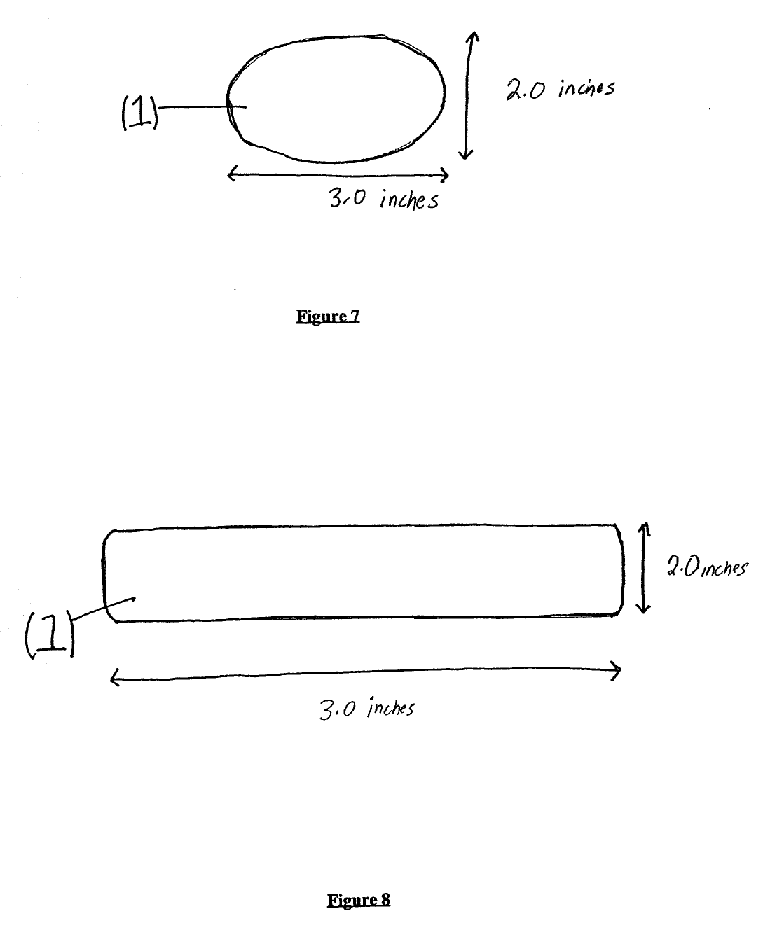

In one specific embodiment of the present invention, the adhesive skin patch 1 can be crescent, oval or circular in shape. The circular adhesive skin patch 1 can have a diameter of about 0.1 inch to about 10 inches. Preferably, the circular adhesive skin patch 1 can have a diameter of about 1.5 inches to about 5 inches. See, FIG. 7.

In another specific embodiment of the present invention, the adhesive skin patch 1 can be rectangular in shape. The rectangular adhesive skin patch 1 can have a length of about 1 inch to about 10 inches and a width of about 1 inch to about 10 inches. Preferably, the rectangular adhesive skin patch 1 can have a length of about 2 inches to about 5 inches and a width of about 2 inches to about 5 inches. See, FIG. 8.

In one embodiment of the present invention, the adhesive skin patch 1 can have a release liner 10 mounted on the front side 3 of the backing 2 of the adhesive skin patch 1. In such an embodiment, one or more adhesive skin patches 1 can be mounted on the release liner 10. For example, one adhesive skin patch 1 can have one release liner 10 mounted on the front side 3 of the backing 2 of the adhesive skin patch 1. Alternatively, about 2 to about 100 or about 2 to about 20 adhesive skin patches 1 can be mounted on the release liner 10. The cost of having two or more patches 1 on a single release liner 10 is typically less expensive than skin patches 1 that are separately mounted on a single release liner 10. In addition, some consumers may prefer the ease and comfort of carrying a single patch assembly that includes a single release liner 10 and more than one (e.g., about 2 to about 20, or about 2 to about 10) adhesive patches 1 mounted on the single release liner 10.

The adhesive skin patch 1 can be applied to the region of the anus of a patient. As used herein, the “region of the anus” refers to the anal canal, the perianal area, the lower rectal areas and the topical (exterior) surface of the anus.

The adhesive patch 1 serves as a protective covering or barrier. Such protection serves to prevent or diminish the likelihood that foreign objects (e.g., a person's clothing, etc.) will come into contact with the hemorrhoidal tissue. This may effectively decrease the likelihood that the hemorrhoidal tissue will become further irritated or infected.

The adhesive patch 1 also serves to absorb exudate, blood, or a combination thereof that is typically accompanied with hemorrhoids. As such, the use of the adhesive patch 1 of the present invention can allow those individuals inflicted with hemorrhoids to go out in public without the fear, embarrassment, and/or inconvenience of having a bloody anus that may soil the individual's clothing.

The adhesive patch 1 of the present invention can be formulated or manufactured employing the above components. The adhesive patch 1 of the present invention can be formulated or manufactured using any suitable technique. Preferably, the adhesive patch 1 can be formulated or manufactured as described herein or as described in U.S. Pat. No. 5,536,263; U.S. Pat. No. 5,741,510; and references cited therein; wherein the oil premix includes the vasoconstrictor 15, propylene glycol, and solvent 13; the glycerin premix includes glycerin, Vitamin E, and aloe vera gel; and the adhesive premix includes the adhesive, polymer 9, and water; and wherein the backing can be treated with a sizing agent 8 prior to the infusion of the therapeutic formulation 5.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. An adhesive patch comprising a flexible backing having a front side and a back side and a therapeutic formulation positioned on at least a portion of the front side of the backing, in at least a portion of the front side of the backing, or on and in at least a portion of the front side of the backing; wherein the therapeutic formulation comprises:
a vasoconstrictor;
a solvent that dissolves the vasoconstrictor; and
a pressure sensitive adhesive.

2. The adhesive patch of claim 1 wherein the vasoconstrictor is ephedrine, epinephrine, phenylephrine, a pharmaceutically acceptable salt thereof, or a combination thereof.

3. The adhesive patch of claim 1 wherein the vasoconstrictor is ephedrine sulfate, epinephrine, epinephrine hydrochloride, phenylephrine hydrochloride, or a combination thereof.

4. The adhesive patch of claim 3 wherein ephedrine sulfate is present in up to about 1.25 wt. % of the therapeutic formulation; the epinephrine is present up to about 0.01 wt. % of the therapeutic formulation; the epinephrine hydrochloride is present up to about 0.01 wt. % of the therapeutic formulation; or the phenylephrine hydrochloride is present up to about 0.30 wt. % of the therapeutic formulation.

5. The adhesive patch of claim 3 wherein ephedrine sulfate is present in about 0.1 wt. % of the therapeutic formulation to about 1.25 wt. % of the therapeutic formulation; the epinephrine is present in about 0.005 wt. % of the therapeutic formulation to about 0.01 wt. % of the therapeutic formulation; the epinephrine hydrochloride is present in about 0.005 wt. % of the therapeutic formulation to about 0.01 wt. % of the therapeutic formulation; or the phenylephrine hydrochloride is present in about 0.20 wt. % of the therapeutic formulation to about 0.30 wt. % of the therapeutic formulation.

6. The adhesive patch of claim 1 wherein the vasoconstrictor is located on the entire surface of the front side of the backing.

7. The adhesive patch of claim 1 wherein the vasoconstrictor is at least partially embedded in the front side of the backing.

8. The patch of claim 1 wherein the vasoconstrictor is completely embedded in the backing.

9. The adhesive patch of claim 1 wherein the solvent comprises a polyhydric alcohol, water, or a combination thereof.

10. The adhesive patch of claim 9 wherein the polyhydric alcohol is glycerin, propylene glycol, ethylene glycol, triethylene glycol, or a combination thereof.

11. The adhesive patch of claim 9 wherein the polyhydric alcohol is present up to about 70 wt. % of the therapeutic formulation.

12. The adhesive patch of claim 9 wherein the polyhydric alcohol is present in about 20 wt. % to about 60 wt. % of the therapeutic formulation.

13. The adhesive patch of claim 9 wherein the water is present in about 2 wt. % to about 50 wt. % of the therapeutic formulation.

14. The adhesive patch of claim 1 wherein the solvent is present in about 5 wt. % to about 30 wt. % of the therapeutic formulation.

15. The adhesive patch of claim 1 wherein the solvent comprises water; triethylene glycol; glycerin; propylene glycol; ethylene glycol; triacetin; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; diethylene glycol methyl ether; diethylene glycol methyl ether acetate; a pharmaceutically acceptable salt thereof; or a combination thereof.

16. The adhesive patch of claim 1 wherein the solvent is located on the entire surface of the front side of the backing.

17. The adhesive patch of claim 1 wherein the solvent is at least partially embedded in the front side of the backing.

18. The patch of claim 1 wherein the solvent is completely embedded in the backing.

19. The adhesive patch of claim 1 wherein the pressure sensitive adhesive comprises one or more acrylic ester copolymers.

20. The adhesive patch of claim 19 wherein each of the one or more acrylic ester copolymers is present up to about 30 wt. % of the therapeutic formulation.

21. The adhesive patch of claim 19 wherein each of the one or more acrylic ester copolymers is present in about 2 wt. % of the therapeutic formulation to about 30 wt. % of the therapeutic formulation.

22. The adhesive patch of claim 19 wherein all of the one or more acrylic ester copolymers, combined, are present in about 2 wt. % to about 30 wt. % of the therapeutic formulation.

23. The adhesive patch of claim 1 wherein the pressure sensitive adhesive is located on the entire surface of the front side of the backing.

24. The adhesive patch of claim 1 wherein the pressure sensitive adhesive is at least partially embedded in the front side of the backing.

25. The patch of claim 1 wherein the pressure sensitive adhesive is completely embedded in the backing.

26. The adhesive patch of claim 1 wherein the pressure sensitive adhesive further comprises an emulsifier.

27. The adhesive patch of claim 26 wherein the emulsifier is pectin.

28. The adhesive patch of claim 27 wherein the pectin is present in about 2.0 wt. % to about 20.0 wt. % of the therapeutic formulation.

29. The adhesive patch of claim 1 wherein the therapeutic formulation further comprises a compound that provides structure and strength to the pressure sensitive adhesive or to the therapeutic formulation.

30. The adhesive patch of claim 29 wherein the compound that provides structure and strength to the pressure sensitive adhesive or to the therapeutic formulation is karaya, a polyacrylamide, xanthum gum, guar gum, a natural polymer, a synthetic polymer, a hydrophilic polymer, a hydrocolloidal polymer, starch, a starch derivative or graft copolymer, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, or a mixture thereof.

31. The adhesive patch of claim 29 wherein the compound that provides structure and strength to the pressure sensitive adhesive or provides structure and strength to the therapeutic formulation is polyacrylamide.

32. The adhesive patch of claim 31 wherein the polyacrylamide is present in up to about 25 wt. % of the therapeutic formulation.

33. The adhesive patch of claim 31 wherein the polyacrylamide is present in about 1 wt. % to about 25 wt. % of the therapeutic formulation.

34. The adhesive patch of claim 29 wherein the compound that provides structure and strength to the pressure sensitive adhesive or provides structure and strength to the therapeutic formulation is karaya.

35. The adhesive patch of claim 34 wherein the karaya is present in about 1 wt. % to about 30 wt. % of the therapeutic formulation.

36. The adhesive patch of claim 29 wherein the compound that provides structure and strength to the pressure sensitive adhesive or provides structure and strength to the therapeutic formulation is a combination of polyacrylamide and karaya.

37. The adhesive patch of claim 1 wherein the therapeutic formulation is partially embedded in at least a portion of the front side of the backing.

38. The adhesive patch of claim 1 wherein the therapeutic formulation is located on the entire surface of the front side of the backing.

39. The adhesive patch of claim 1 wherein the backing is porous.

40. The adhesive patch of claim 1 wherein the backing is vapor permeable.

41. The adhesive patch of claim 1 wherein the backing comprises water insoluble material.

42. The adhesive patch of claim 1 wherein the backing comprises water soluble material.

43. The adhesive patch of claim 1 wherein the backing comprises polycellulose fibers, polyester fibers, polyurethane fibers, polyolefin fibers, polyamide fibers, cotton fibers, copolyester fibers, or any mixture thereof.

44. The adhesive patch of claim 1 wherein upon contact with skin, the backing retains the therapeutic formulation and the patch allows moisture from the skin to pass.

45. The adhesive patch of claim 1 wherein the backing has a thickness of about 0.025 mm to about 1.25 mm.

46. The adhesive patch of claim 1 wherein the backing comprises a nonwoven fabric.

47. The adhesive patch of claim 1 wherein at least a portion of the backing is treated with a sizing agent such that the portion of the backing that is treated with the sizing agent has a surface energy of about 20 dynes/cm² to about 65 dynes/cm².

48. The adhesive patch of claim 47 wherein the sizing agent is a fluorocarbon solution, a silicone-containing compound, or a combination thereof.

49. The adhesive patch of claim 48 wherein the backing that is treated with the fluorocarbon solution is Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, Vilmed M1578 FH, or a combination thereof.

50. The adhesive patch of claim 48 wherein the silicone-containing compound is a polydimethyl siloxane, a dialkylsiloxane, a dimethylsiloxo vinyl alkene, a dialkylsiloxo vinyl alkene, a dimethylsiloxo acrylate, a dialkylsiloxo acrylate, a vinyl terminated polydimethylsiloxane, a vinyl terminated polydialkylsiloxane, or a combination thereof.

51. The adhesive patch of claim 47 wherein the entire front side of the backing is treated with the sizing agent.

52. The adhesive patch of claim 47 wherein the sizing agent penetrates at least a portion of the underlying surface of the front side of the backing.

53. The adhesive patch of claim 47 wherein the sizing agent penetrates the entire underlying surface of the front side of the backing.

54. The adhesive patch of claim 47 wherein the entire backing is treated with the sizing agent.

55. The adhesive patch of claim 1 wherein the therapeutic formulation further comprises an analgesic, an anesthetic, an antipruritic, or a combination thereof.

56. The adhesive patch of claim 55 wherein the analgesic, the anesthetic, the antipruritic, or the combination thereof is camphor, menthol, benzocaine, butamben picrate, dibucaine, dimethisoquin, dyclonine, lidocaine, pramoxine, tetracaine, benzyl alcohol, camphorated metacresol, juniper tar, phenol, resorcinol, diphenhydramine, tripelennamine, hydrocortisone, hydrocortisone acetate, a pharmaceutically acceptable salt thereof, or a combination thereof.

57. The adhesive patch of claim 55 wherein the analgesic, the anesthetic, the antipruritic, or the combination thereof is camphor, menthol, benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphorated metacresol, juniper tar, phenol, phenolate sodium, resorcinol, diphenhydramine hydrochloride, tripelennamine hydrochloride, hydrocortisone, hydrocortisone acetate, or a combination thereof.

58. The adhesive patch of claim 57 wherein the camphor is present up to about 3.0 wt. % of the therapeutic formulation and menthol is present up to about 1.0 wt. % of the therapeutic formulation; benzocaine is present up to about 20.0 wt. % of the therapeutic formulation; butamben picrate is present up to about 1.5 wt. % of the therapeutic formulation; dibucaine is present up to about 1.0 wt. % of the therapeutic formulation; dibucaine hydrochloride is present up to about 1.0 wt. % of the therapeutic formulation; dimethisoquin hydrochloride is present up to about 0.5 wt. % of the therapeutic formulation; dyclonine hydrochloride is present up to about 1.0 wt. % of the therapeutic formulation; lidocaine is present up to about 4.0 wt. % of the therapeutic formulation; lidocaine hydrochloride is present up to about 4.0 wt. % of the therapeutic formulation; pramoxine hydrochloride is present up to about 1.0 wt. % of the therapeutic formulation; tetracaine is present up to about 2.0 wt. % of the therapeutic formulation; tetracaine hydrochloride is present up to about 2.0 wt. % of the therapeutic formulation; benzyl alcohol is present up to about 33.0 wt. % of the therapeutic formulation; camphor is present up to about 3.0 wt. % of the therapeutic formulation; juniper tar is present up to about 5.0 wt. % of the therapeutic formulation; phenolate sodium is present up to about 1.5 wt. % of the therapeutic formulation; resorcinol is present up to about 3.0 wt. % of the therapeutic formulation; diphenhydramine hydrochloride is present up to about 2.0 wt. % of the therapeutic formulation; tripelennamine hydrochloride is present up to about 2.0 wt. % of the therapeutic formulation; hydrocortisone is present up to about 1.0 wt. % of the therapeutic formulation; corticosteroid is present up to about 5.0 wt. % of the therapeutic formulation; camphor is present up to about 10.8 wt. % of the therapeutic formulation with phenol; camphor is present up to about 10.8 wt. % of the therapeutic formulation with metacresol in about 1 wt. % to about 3.6 wt. % of the therapeutic formulation, as camphorated metacresol; or hydrocortisone acetate is present up to about 1.0 wt. % of the therapeutic formulation.

59. The adhesive patch of claim 57 wherein the camphor is present up to about 3.0 wt. % of the therapeutic formulation and menthol is present up to about 1.0 wt. % of the therapeutic formulation; benzocaine is present in about 5.0 wt. % to about 20.0 wt. % of the therapeutic formulation; butamben picrate is present in about 0.5 wt. % to about 1.5 wt. % of the therapeutic formulation; dibucaine is present in about 0.25 wt. % to about 1.0 wt. % of the therapeutic formulation; dibucaine hydrochloride is present in about 0.25 wt. % to about 1.0 wt. % of the therapeutic formulation; dimethisoquin hydrochloride is present in about 0.3 wt. % to about 0.5 wt. % of the therapeutic formulation; dyclonine hydrochloride is present in about 0.5 wt. % to about 1.0 wt. % of the therapeutic formulation; lidocaine is present in about 0.5 wt. % to about 4.0 wt. % of the therapeutic formulation; lidocaine hydrochloride is present in about 0.5 wt. % to about 4.0 wt. % of the therapeutic formulation; pramoxine hydrochloride is present in about 0.5 wt. % to about 1.0 wt. % of the therapeutic formulation; tetracaine is present in about 1.0 wt. % to about 2.0 wt. % of the therapeutic formulation; tetracaine hydrochloride is present in about 1.0 wt. % to about 2.0 wt. % of the therapeutic formulation; benzyl alcohol is present in about 10.0 wt. % to about 33.0 wt. % of the therapeutic formulation; camphor is present in about 0.1 wt. % to about 3.0 wt. % of the therapeutic formulation; juniper tar is present in about 1.0 wt. % to about 5.0 wt. % of the therapeutic formulation; phenolate sodium is present in about 0.5 wt. % to about 1.5 wt. % of the therapeutic formulation; resorcinol is present in about 0.5 wt. % to about 3.0 wt. % of the therapeutic formulation; diphenhydramine hydrochloride is present in about 1.0 wt. % to about 2.0 wt. % of the therapeutic formulation; tripelennamine hydrochloride is present in about 0.5 wt. % to about 2.0 wt. % of the therapeutic formulation; hydrocortisone is present in about 0.25 wt. % to about 1.0 wt. % of the therapeutic formulation; corticosteroid is present in about 0.25 to about 5.0 wt. % of the therapeutic formulation; camphor is present in about 3 wt. % to about 10.8 wt. % of the therapeutic formulation with phenol; camphor is present in about 3 wt. % to about 10.8 wt. % of the therapeutic formulation with metacresol in about 1 wt. % to about 3.6 wt. % of the therapeutic formulation, as camphorated metacresol; or hydrocortisone acetate is present in about 0.25 wt. % to about 1.0 wt. % of the therapeutic formulation.

60. The adhesive patch of claim 57 wherein the hydrocortisone, the hydrocortisone acetate, or the combination thereof is present in about 0.25 wt. % to about 1.0 wt. % of the therapeutic formulation; the lidocaine, lidocaine hydrochloride, or the combination thereof is present in about 0.5 wt. % to about 4.0 wt. % of the therapeutic formulation; the camphor is present in about 0.1 wt. % of the therapeutic formulation to about 3.0 wt. % of the therapeutic formulation; the juniper tar is present in about 1.0 wt. % of the therapeutic formulation to about 5.0 wt. % of the therapeutic formulation; or the menthol is present in about 0.1 wt. % of the therapeutic formulation to about 1.0 wt. % of the therapeutic formulation.

61. The adhesive patch of claim 57 wherein the benzocaine is present in about 5.0 wt. % of the therapeutic formulation to about 20.0 wt. % of the therapeutic formulation; the benzyl alcohol is present in about 1.0 wt. % of the therapeutic formulation to about 4.0 wt. % of the therapeutic formulation; the dibucaine is present in about 0.25 wt. % of the therapeutic formulation to about 1.0 wt. % of the therapeutic formulation; the dibucaine hydrochloride is present in about 0.25 wt. % of the therapeutic formulation to about 1.0 wt. % of the therapeutic formulation; the dyclonine hydrochloride is present in about 0.5 wt. % of the therapeutic formulation to about 1.0 wt. % of the therapeutic formulation; the lidocaine is present in about 2.0 wt. % of the therapeutic formulation to about 5.0 wt. % of the therapeutic formulation; the pramoxine hydrochloride is present in about 0.5 wt. % of the therapeutic formulation to about 1.5 wt. % of the therapeutic formulation; the tetracaine is present in about 0.5 wt. % of the therapeutic formulation to about 1.0 wt. % of the therapeutic formulation; or the tetracaine hydrochloride is present in about 0.5 wt. % of the therapeutic formulation to about 1.0 wt. % of the therapeutic formulation.

62. The adhesive patch of claim 1 wherein the therapeutic formulation further comprises an anti-inflammatory agent.

63. The adhesive patch of claim 1 wherein the anti-inflammatory agent is a corticosteroid.

64. The adhesive patch of claim 63 wherein the corticosteroid is at least one of cortisol (hydrocortisone); tetrahydrocortisol; prednisone (cortisone); prednisolone (cortisol); 6α-methylprednisolone; fludrocortisone (9α-fluorocortisol); 11-desoxycortisol; cortisone (11-dehydrocortisol); corticosterone; triamcinolone (9α-fluoro-16α-hydroxyprednisolone); paramethasone (6α-fluoro-16α-methylprednisolone); betamethasone (9α-fluoro-16β-methylprednisolone); dexamethasone (9α-fluoro-16α-methylprednisolone); desoxycorticosterone acetate (doca acetate, percorten acetate); desoxycorticosterone pivalate (percorten pivalate); fludrocortisone acetate (florine acetate); cortisol (hydrocortisone) (cortef, hydrocortone); cortisol acetate (cortef acetate, hydrocortone acetate); cortisol cypionate (cortef); cortisol sodium phosphate (hydrocortone phosphate); cortisol sodium succinate (solu-cortef); beclopmethasone dipropionate (vanceril); betamethasone (celestone); betamethasone sodium phosphate and acetate (celestone soluspan); betamethasone dipropionate (diprosone); betamethasone valerate (valisone); betamethasone benzoate (benisone, flurodate); cortisone acetate (cortone acetate); dexamethasone (decadron, gammacorten); dexamethasone sodium phosphate (decadron phosphate, hexadrol phosphate); dexamethasone acetate (decadron-L.A.); fuprednisolone (alphadrol); meprednisone (betapar); methylprednisolone (medrol); methylprednisolone acetate (depo-medrol, medrol acetate); methylprednisolone sodium succinate (solu-medrol); paramethasone acetate (haldrone); prednisolone (delta-cortef); prednisolone acetate (meticortelone acetate); prednisolone sodium phosphate (hydeltrasol); prednisolone sodium succinate (meticortelone soluble); prednisolone tebutate (hydelta-T.B.A.); prednisone (deltasone, paracort); triamcinolone (aristocort, kenacort); triamcinolone acetonide (aristoderm, kenalog); triamcinolone diacetate (aristocort diacetate, kienacort diacetate); triamcinolone hexacotonide (aristospan); desonide (tridesilon); desoximetasone (topicort); flumethasone pivalate (locorten); fluocinolone acetonide (fluonid, synalar); fluocinonide (lidex, topsyn); fluorometholone (oxylone); flurandrenolide (cordran); halcinonide (halog); medrysone (HMS liquifilm, medrocort); aclometasone dipropionate (alclovate); betamethasone-17-benzoate (benisone, flurobate); betamethasone dipropionate (diprosone); betamethasone-17-valerate (valisone); clobetasol propionate (temovate); desonide (desowen, tridesilon); dexamethasone (aeroseb-D); desoximetasone (topicort); diflorasone diacetate (florone); flumethasone pivalate (locorten); fluocinolone acetonide (synalar, synalar-HP, neosynalar, fluonid); fluocinolone acetonide acetate (lidex; lidex-E; topsyn); fluorometholone (oxylone); flurandrenolide (cordran); halcinonide (halog); hydrocortisone (cort-dome, lubricort); hydrocortisone acetate (cortef, carmol HC, neo-cortef); hydrocortisone-17-valerate (westcort); prednisolone (meti-derm); triamcinolone acetonide (kenalog, orabase, kenalog-S, mycolog, aristocort, aristocort-A, aristoderm, neo-aristoderm, neo-aristocort); temovate; diprolen; psorcon; temovate; diprolene; cyclocort; diprosone; florone; halog; lidex; maxiflor; topicort; aristocort A; diprosone; florone; maxiflor; valisone; cordran; kenalog; synalar; topicort LP; westcort; cordran; diprosone; kenalog; locold; synalar; valisone; westcort; aclovate; desowen; locorten; synalar; tridesilone; valisone; hydrocortisone; dexamethasone; flumethalone; prednisolone; methylprednisolone; augmented betamethasone dipropionate (diprolene); diflorasone diacetate (psorcon); clobetasol propionate (temovate); halobetasol propionate (ultravate); amcinonide (cyclocort); betamethasone dipropionate (diprolene, diprosone); diflorasone diacetate (florone); halcinonide (halog); fluocinonide (lidex); diflorasone diacetate (maxiflor); betamethasone dipropionate (maxivate); diflorasone diacetate (psorcom); desoximetasone (topicort); (aristocort A); amcinonide (cyclocort); betamethasone dipropionate (diprosone); mometasone furoate (elocon); diflorasone diacetate (florone); halcinonide (halog); fluocinonide (lidex-E); diflorasone diacetate (maxiflor); betamethasone dipropionate (maxivate, psorion); betamethasone valerate (valisone); flurandrenolide (cordran); fluticasone propionate (cutivate); mometasone furoate (elocon); triamcinolone acetonide (kenalog); fluocinolone acetonide (synalar); hydrocortisone valerate (westcort); flurandrenolide (cordran); fluticasone propionate (cutivate); betamethasone dipropionate (diprosone); triamcinolone acetonide (kenalog); hydrocortisone butyrate (locoid); fluocinolone acetonide (synalar); betamethasone valerate (valisone); hydrocortisone valerate (westcort); alclometasone dipropionate (aclovate); triamcinolone acetonide (aristocort); desonide (desowen); flumethasone pivalate (locorten); fluocinolone acetonide (synalar); desonide (tridesilon); betamethasone valerate (valisone); hydrocortisone (eldecort, dexamethasone, flumethalone, hydrocortisone, methylprednisolone, or prednisolone); betamethasone; dexamethasone; and pharmaceutically acceptable salts thereof.

65. The adhesive patch of claim 63 wherein the corticosteroid is present up to about 5 wt. % of the therapeutic formulation.

66. The adhesive patch of claim 1 wherein the therapeutic formulation further comprises one or more skin conditioners.

67. The adhesive patch of claim 66 wherein the skin conditioner is calamine, aluminum hydroxide gel, cocoa butter, aloe, lanolin, glycerin, Vitamin E, Vitamin E acetate, farnesol, glycyrrhetinic acid, propylene glycol, ethylene glycol, triethylene glycol, hard fat, kaolin, lanolin, mineral oil, petrolatum, topical starch, white petroleum, cod liver oil, shark liver oil, zinc oxide; or a combination thereof.

68. The adhesive patch of claim 67 wherein the glycerin is present up to about 45 wt. % of the therapeutic formulation, the aloe is present up to about 2.0 wt. % of the therapeutic formulation; or the Vitamin E acetate is present up to about 2.0 wt. % of the therapeutic formulation.

69. The adhesive patch of claim 1 further comprising an astringent.

70. The adhesive patch of claim 69 wherein the astringent is calamine, witch hazel, zinc oxide, or a combination thereof.

71. The adhesive patch of claim 70 wherein the calamine is present up to about 25 wt. % of the thetapeutic formulation; the witch hazel is present up to about 50 wt. % of the therapeutic formulation; or the zinc oxide is present up to about 25 wt. % of the thetapeutic formulation.

72. The adhesive patch of claim 70 wherein the calamine is present in about 5 wt. % of the therapeutic formulation to about 25 wt. % of the thetapeutic formulation; the witch hazel is present in about 10 wt. % of the therapeutic formulation to about 50 wt. % of the therapeutic formulation; or the zinc oxide is present in about 5 wt. % of the therapeutic formulation to about 25 wt. % of the thetapeutic formulation.

73. The adhesive patch of claim 1 further comprising a keratolytic agent.

74. The adhesive patch of claim 73 wherein the keratolytic agent is alcloxa, resorcinol, or a combination thereof.

75. The adhesive patch of claim 74 wherein the alcloxa is present up to about 2.0 wt. % of the therapeutic formulation; or the resorcinol is present up to about 3.0 wt. % of the thetapeutic formulation.

76. The adhesive patch of claim 74 wherein the alcloxa is present in about 0.2 wt. % of the therapeutic formulation to about 2.0 wt. % of the therapeutic formulation; or the resorcinol is present in about 1.0 wt. % of the therapeutic formulation to about 3.0 wt. % of the thetapeutic formulation.

77. The adhesive patch of claim 1 wherein the therapeutic formulation further comprises a filler.

78. The adhesive patch of claim 77 wherein the filler is malto dextrin.

79. The adhesive patch of claim 78 wherein the malto dextrin is present in about 1.0 wt. % to about 10.0 wt. % of the therapeutic formulation.

80. The adhesive patch of claim 1 having a thickness of about 0.20 mm to about 0.75 mm.

81. The adhesive patch of claim 1 further comprising a release liner that is mounted on the front side of the backing.

82. The adhesive patch of claim 81 wherein more than one patch is mounted on the release liner.

83. The adhesive patch of claim 81 wherein about 2 to about 20 adhesive patches are mounted on the release liner.

84. The adhesive patch of claim 62 wherein the therapeutic formulation further comprises a complexing agent that effectively solubilizes or stabilizes the anti-inflammatory agent.

85. The adhesive patch of claim 84 wherein the complexing agent is a cyclodextrin, or a derivative of cyclodextrin.

86. The adhesive patch of claim 85 wherein the cyclodextrin or the derivative of cyclodextrin is alpha-cyclodextrin, beta-cyclodextrin, gamma-cyclodextrin, alpha-cyclodextrin sulfate, beta-cyclodextrin sulfate, gamma-cyclodextrin sulfate, alpha-hydroxypropyl cyclodextrin, beta-hydroxypropyl cyclodextrin, gamma-hydroxypropyl cyclodextrin, alpha-cyclodextrin phosphate, beta-cyclodextrin phosphate, gamma-cyclodextrin phosphate, or a combination thereof.

87. The adhesive patch of claim 1 wherein the therapeutic formulation further comprises an antibiotic agent.

88. The adhesive patch of claim 87 wherein antibiotic agent is a β-lactam, an aminoglycoside, an antifungal agent, or a combination thereof.

89. The adhesive patch of claim 87 wherein antibiotic agent is cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole, sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, pinworm, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscamet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscamet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, a pharmaceutically acceptable salt thereof, or a combination thereof.

90. A method for treating or preventing hemorrhoids in a mammal in need thereof, the method comprising applying to the region of the anus of the mammal having the hemorrhoid or to the region of the anus of the mammal at risk thereof an adhesive patch of claim 1 for a period of time effective to treat or prevent the hemorrhoid.

91. The method of claim 90 wherein the mammal is a human.

92. The method of claim 90 wherein the period of time is up to about 24 hours.

93. A method for providing relief from the discomfort associated with hemorrhoids, the method comprising applying to the region of the anus of a mammal having the hemorrhoid an adhesive patch of claim 1 for a period of time effective to provide the relief.

94. The method of claim 93 wherein the discomfort includes burning, itching, swelling, irritation, soreness, inflammation, or a combination thereof, of hemorroidal tissue.

95. The method of claim 93 wherein the mammal is a human.

96. The method of claim 93 wherein the period of time is up to about 24 hours.

97. A method for providing post-operative relief from discomfort associated with a surgical treatment of hemorrhoids, the method comprising applying to the region of the anus of the mammal an adhesive patch of claim 1 for a period of time effective to provide the relief.

98. The method of claim 97 wherein the mammal is a human.

99. The method of claim 97 wherein the period of time is up to about 24 hours.

100. A method for treating or preventing a bacterial infection associated with hemorrhoids, the method comprising applying to the region of the anus of a mammal having the hemorrhoid or to the region of the anus of a mammal at risk thereof an adhesive patch of claim 1 for a period of time effective to treat or prevent the bacterial infection.

101. The method of claim 100 wherein the mammal is a human.

102. The method of claim 100 wherein the period of time is up to about 24 hours.

103. A method for treating or preventing a bacterial infection associated with the surgical treatment of hemorrhoids, the method comprising applying to the region of the anus of the mammal an adhesive patch of claim 1 for a period of time effective to treat or prevent the bacterial infection.

104. The method of claim 103 wherein the mammal is a human.

105. The method of claim 103 wherein the period of time is up to about 24 hours.

106. A method for absorbing exudate, blood, or a combination thereof from the region of the anus of a mammal inflicted with hemorrhoids, the method comprising applying to the region of the anus of the mammal an adhesive patch of claim 1 for a period of time effective to absorb the exudate, blood, or the combination thereof.

107. The method of claim 106 wherein the mammal is a human.

108. The method of claim 106 wherein the period of time is up to about 24 hours.

109. A method for absorbing exudate, blood, or a combination thereof from the region of the anus of a mammal during the post-operative treatment of hemorrhoids, the method comprising applying to the region of the anus of the mammal an adhesive patch of claim 1 for a period of time effective to absorb the exudate, blood, or the combination thereof.

110. The method of claim 109 wherein the mammal is a human.

111. The method of claim 109 wherein the period of time is up to about 24 hours.
